SEQUENCE LISTING (11 sequences)

SEQ ID NO: 1 - protein, 448 aa, synthetic construct
QIQLVQSGSELKKPGASVKVSCKASGYTFTTAGMQWVRQAPGQGLEWIGWINSHSGVPKYAEDFKGRFVFSLDTSVSTAYLQISSLKAEDTAVYFCARGGDEGVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSFLSASVGDRVTITCKASQDVSTAVAWYQQKPGQPPKLLIYWTSTRHTGVPDRFSGSGSGTDYTLTISSLQAEDVAVYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 107 aa, synthetic construct
DIQMTQSPSFLSASVGDRVTITCKASQDVSTAVAWYQQKPGQPPKLLIYWTSTRHTGVPDRFSGSGSGTDYTLTISSLQAEDVAVYYCQQHYSTPWTFGGGTKVEIK

SEQ ID NO: 4 - protein, 107 aa, synthetic construct
DIVMTQSPSFLSASVGDRVTITCKASQDVSTAVAWYQQKPGQPPKLLIYWTSTRHTGVPDRFSGSGSGTDYTLTISSVQAEDVAVYYCQQHYSTPWTFGGGTKVEIK

SEQ ID NO: 5 - protein, 118 aa, synthetic construct
QIQLVQSGSELKKPGASVKVSCKASGYTFTTAGMQWVRQAPGQGLEWIGWINSHSGVPKYAEDFKGRFVFSLDTSVSTAYLQISSLKAEDTAVYFCARGGDEGVMDYWGQGTTVTVSS

SEQ ID NO: 6 - protein, 5 aa, Mus musculus
TAGMQ

SEQ ID NO: 7 - protein, 16 aa, Mus musculus
WINSHSGVPKYAEDFK

SEQ ID NO: 8 - protein, 9 aa, Mus musculus
GGDEGVMDY

SEQ ID NO: 9 - protein, 11 aa, Mus musculus
KASQDVSTAVA

SEQ ID NO: 10 - protein, 7 aa, Mus musculus
WTSTRHT

SEQ ID NO: 11 - protein, 9 aa, Mus musculus
QQHYSTPWT

(19) United States
(12) Patent Application Publication
(10) Pub. No.: US 2026/0007744 A1
(43) Pub. Date: Jan. 8, 2026

(54) STABLE AQUEOUS FORMULATIONS FOR ANTIBODIES

(71) Applicant: INNATE PHARMA, Marseille (FR)

(72) Inventors: Thibault Barbier, Marseille (FR); Angélique Boedec-Herbette, Marseille (FR); Laetitia Cohen-Tannoudji, Marseille (FR)

(21) Appl. No.: 19/242,578

(22) Filed: Jun. 18, 2025

Related U.S. Application Data
(60) Provisional application No. 63/661,466, filed on Jun. 18, 2024.

(51) Int. Cl. A61K 39/395; A61K 9/08; A61K 47/02; A61K 47/26; C07K 16/28

(52) U.S. Cl. CPC A61K 39/39591; A61K 9/08; A61K 47/02; A61K 47/26; C07K 16/2803

(57) ABSTRACT

The present disclosure relates to a stable aqueous formulation. The stable aqueous formulation may be used for an anti-KIR3DL2 antibody.

### CROSS REFERENCE TO RELATED APPLICATION

This application claims the benefit of U.S. Provisional Patent Application Ser. No. 63/661,466, filed Jun. 18, 2024. The entire content of the above-referenced patent application is incorporated by reference in its entirety herein.

### REFERENCE TO SEQUENCE LISTING

The instant application contains a Sequence Listing which has been submitted electronically in XML format and is hereby incorporated by reference in its entirety. Said XML file, created on Jun. 18, 2025, is named 766848_INN9-001_ST26.xml and is 11,433 bytes in size.

### FIELD

The present disclosure relates to stable antibody formulations. More specifically, the disclosure relates to formulations for reducing or minimizing particle formation.

### BACKGROUND

Antibodies are a leading group of therapeutics. However, such therapeutics encounter various stresses during the development, manufacture, and clinical use. The stresses can affect the therapeutic's attributes by forming particles, aggregation or denaturing the therapeutic.

### SUMMARY

Disclosed are formulations for providing a stable aqueous formulation for the anti-KIR3DL2 antibodies.

In one aspect is provided a stable aqueous pharmaceutical formulation comprising: (i) an anti-KIR3DL2 antibody that binds to a KIR3DL2 receptor; (ii) one or more buffers; (iii) a tonicity agent; and (iv) a non-ionic surfactant; wherein the ratio of the antibody concentration to non-ionic surfactant is about 20:0.1 to 40:0.4.

In certain exemplary embodiments, the antibody comprises a heavy chain variable region (VH) and a light chain variable region (VL), wherein the VH comprises three complementarity determining regions (CDRs) CDR1, CDR2, and CDR3 amino acid sequences set forth in SEQ ID NOs: 6, 7 and 8, respectively and wherein the VL comprises three CDRs, CDR1, CDR2, and CDR3 amino acid sequences set forth in SEQ ID NOs: 9, 10 and 11, respectively. In certain exemplary embodiments, the antibody comprises a VH and VL region comprising the amino acid sequence of SEQ ID NOS: 5 and 3 respectively.

In certain exemplary embodiments, the antibody concentration is at least 15 mg/ml.

In certain exemplary embodiments, the pharmaceutical formulation comprises one or more buffers, wherein the buffer is selected from the group consisting of acetate, carbonate, citrate, glycylglycine, histidine, glycine, lysine, arginine, phosphates, or any combination thereof. In certain exemplary embodiments, the buffer is a phosphate buffer selected from the group consisting of sodium dihydrogen phosphate, disodium hydrogen phosphate, sodium phosphate, or any combination thereof. In certain exemplary embodiments, the phosphate buffer is a dibasic sodium phosphate and monobasic sodium phosphate.

In certain exemplary embodiments, the tonicity agent is selected from a salt, a sugar or sugar alcohol, an amino acid, an alditol, polyethyleneglycol, or any combination thereof. In certain exemplary embodiments, the tonicity agent comprises sodium chloride.

In certain exemplary embodiments, the surfactant is a non-ionic surfactant. In certain exemplary embodiments, the nonionic surfactant is selected from the group consisting of polyoxyethylene sorbitan fatty acid esters, polyoxyethylene alkyl ethers, alkylphenylpolyoxyethylene ethers, polyoxyethylene-polyoxypropylene copolymer and combinations thereof. In certain exemplary embodiments, the nonionic surfactant is polysorbate. In certain exemplary embodiments, the polysorbate is polysorbate 80.

In certain exemplary embodiments, the formulation has a pH of about 6.9-7.9, optionally 6.9 to 7.2. In certain exemplary embodiments, the formulation has a pH of about 7.2.

In certain exemplary embodiments, the ratio of the antibody concentration to non-ionic surfactant is about 30:0.2.

In certain exemplary embodiments, the formulation comprises: (i) dibasic sodium phosphate (ii) monobasic sodium phosphate; (iii) sodium chloride; and (iv) polysorbate 80 at a concentration of 0.02-0.2 mg/ml.

In certain exemplary embodiments, the formulation is prepared for intravenous administration.

In another aspect is a stable aqueous pharmaceutical formulation comprising: (i) an anti-KIR3DL2 antibody that binds to a KIR3DL2 receptor at a concentration of at least 15 mg/ml; (ii) polysorbate 80 at a concentration of 0.02-0.2 mg/ml; wherein the formulation further comprises: (a) dibasic sodium phosphate; (b) monobasic sodium phosphate; (c) sodium chloride; and wherein the formulation has less than or equal to 200 particles of ≥10 μm diameter per mL after storage for at least 6, months followed by being subjected to shear or interfacial stress.

In certain exemplary embodiments, the antibody comprises a heavy chain variable region (VH) and a light chain variable region (VL), wherein the VH comprises three complementarity determining regions, CDR1, CDR2, and CDR3 amino acid sequences set forth in SEQ ID NOs: 6, 7 and 8, respectively and wherein the VL comprises three CDRs, CDR1, CDR2, and CDR3 amino acid sequences set forth in SEQ ID NOs: 9, 10 and 11, respectively. In certain exemplary embodiments, the antibody comprises a VH and VL region comprising the amino acid sequence of SEQ ID NOS: 5 and 3 respectively.

In certain exemplary embodiments, the antibody concentration comprises 10 mg/ml to 30 mg/ml, 10 mg/ml to 20 mg/ml, or 12 mg/ml to 18 mg/ml.

In certain exemplary embodiments, the polysorbate 80 comprises about 0.01 mg/ml to about 0.2 mg/ml. In certain exemplary embodiments, the polysorbate 80 comprises 0.1 mg/ml.

In certain exemplary embodiments, the formulation has a pH of about 6.9-7.9. In certain exemplary embodiments, the formulation has a pH of about 7.2.

In certain exemplary embodiments, the formulation is stored for 6 months to 48 months. In certain exemplary embodiments, the formulation is stored at a temperature above 3° C. In certain exemplary embodiments, the formulation is stored at a temperature between 3-8° C.

In certain exemplary embodiments, the particles ≥10 μm comprises no more than 6000 particles per container. In certain exemplary embodiments, the particles <25 μm comprises less than 600 particles per container. In certain exemplary embodiments, the particles size is determined by micro flow imaging, size exclusion chromatograph or visual inspection.

In certain exemplary embodiments, the shear or interfacial stress comprises passage through a syringe.

In yet another aspect is a storage-stable pre-filled syringe comprising an aqueous formulation comprising: (i) an anti-KIR3DL2 antibody that binds to a KIR3DL2 receptor at a concentration of at least 15 mg/ml; (ii) a non-ionic surfactant at a concentration of 0.02-0.2 mg/ml; wherein the formulation further comprises: (a) one or more buffers; and (b) a tonicity agent.

In certain exemplary embodiments, the one or more buffers is selected from the group consisting of acetate, carbonate, citrate, glycylglycine, histidine, glycine, lysine, arginine, phosphates, or any combination thereof. In certain exemplary embodiments, the one or more buffers is a phosphate buffer selected from the group consisting of sodium dihydrogen phosphate, disodium hydrogen phosphate, sodium phosphate, or any combination thereof. In certain exemplary embodiments, the phosphate buffer is a dibasic sodium phosphate and monobasic sodium phosphate.

In certain exemplary embodiments, the tonicity agent is selected from salt, a sugar or sugar alcohol, an amino acid, an alditol, polyethylene glycol, or any combination thereof. In certain exemplary embodiments, the tonicity agent comprises sodium chloride.

In certain exemplary embodiments, the nonionic surfactant is polysorbate. In certain exemplary embodiments, the polysorbate is polysorbate 80.

In certain exemplary embodiments, the formulation has a pH of about 6.9-7.9. In certain exemplary embodiments, the formulation has a pH of about 7.2.

In one aspect is provided a method of reducing stress-induced particle formation or aggregation during drug pooling comprising:
mixing a non-ionic surfactant with a stored antibody formulation to provide a stabilized antibody formulation comprising a ratio of antibody to non-ionic surfactant of 20:0.1 to 40:0.4; the antibody formulation having been stored for at least 6 months;
   whereby the stabilized antibody formulation comprises a reduced amount of particles or aggregated antibody after pooling than the stored antibody formulation after pooling with no additional non-ionic surfactant.

In certain exemplary embodiments, the mixing comprises pumping or passing through a syringe used for IV infusion.

In certain exemplary embodiments, the ratio of the antibody concentration to non-ionic surfactant is about 30:0.2.

In certain exemplary embodiments, the antibody a heavy chain and light chain comprising the amino acid sequence of SEQ ID NOS: 1 and 2 respectively.

In certain exemplary embodiments, the antibody concentration comprises 10 mg/ml to 30 mg/ml, 10 mg/ml to 20 mg/ml, or 12 mg/ml to 18 mg/ml.

In certain exemplary embodiments, the non-ionic surfactant is a polysorbate 80. In certain exemplary embodiments, the polysorbate 80 comprises about 0.01 mg/ml to about 0.2 mg/ml.

In certain exemplary embodiments, the polysorbate 80 comprises 0.1 mg/ml.

In certain exemplary embodiments, the formulation has a pH of about 6.9-7.9. In certain exemplary embodiments, the formulation has a pH of about 7.2.

In certain exemplary embodiments, the formulation is stored for 6 months to 48 months.

In certain exemplary embodiments, the formulation is stored at a temperature above 3° C.

In certain exemplary embodiments, the formulation is stored at a temperature between 3-8° C.

In certain exemplary embodiments, the particles ≥10 μm comprises no more than 6000 particles per container.

In certain exemplary embodiments, the particles <25 μm comprises less than 600 particles per container.

In certain exemplary embodiments, the particles size is determined by micro flow imaging, size exclusion chromatograph or visual inspection.

In certain exemplary embodiments, the stabilized antibody formulation comprises 10-fold less particles or aggregated antibody than the stored antibody formulation after storage for at least 6 months followed by being subjected to shear or interfacial stress, optionally wherein the non-stabilized antibody formulation is a formulation having a ratio of the antibody concentration to non-ionic surfactant of less than 20:0.1.

In certain exemplary embodiments, the stabilized antibody formulation has particulate matter that is less than or equal to 200, particles of ≥10 μm diameter per mL after storage at around + for at least 6, months followed by being subjected to shear or interfacial stress.

In certain exemplary embodiments, the stabilized antibody formulation has particulate matter that is less than or equal to 50, particles of ≥25 μm diameter per mL after storage at around +5° C. (e.g. +5±3° C.) for at least 6, months followed by being subjected to shear or interfacial stress.

In certain exemplary embodiments, the stabilized antibody formulation is stored at around +5° C. (e.g. +5±3° C.).

In certain exemplary embodiments, the particle sizes are assessed by micro flow imaging (MFI). In certain exemplary embodiments, the stabilized antibody formulation has particulate matter as assessed by micro flow imaging (MFI) that is less than or equal to 100 or less than equal to 50 particles of ≥10 μm diameter per mL after storage at around +5° C. (e.g. +5±3° C.) for at least 12 months.

In certain exemplary embodiments, the stabilized antibody formulation has particulate matter as assessed by micro flow imaging (MFI) that is less than or equal to 10 particles of ≥25 μm diameter per mL after storage at around +5° C. (e.g. +5±3° C.) for at least 12 months.

In certain exemplary embodiments, the stabilized antibody formulation comprises less than 1000 particles per mL.

In certain exemplary embodiments, the stabilized antibody formulation is essentially free of visible particles.

In certain exemplary embodiments, the stress is selected from the group consisting of shear stress, interfacial stress, heat stress, and pressure stress, or a combination thereof. In certain exemplary embodiments, the shear stress or interfacial stress is caused by passage through a syringe.

In yet another aspect is provided a method of treating comprising: administering to a subject in need thereof an effective amount of a pharmaceutical formulation comprising: (i) an anti-KIR3DL2 antibody that binds to a KIR3DL2 receptor at a concentration of at least 15 mg/ml; (ii) polysorbate 80 at a concentration of 0.02-0.2 mg/ml; wherein the formulation further comprises: (a) one or more buffers; and (b) a tonicity agent.

In certain exemplary embodiments, the subject in need thereof comprises malignancies involving CD4+ T cells that express KIR3DL2 receptors.

In certain exemplary embodiments, the subject in need therefor comprises cutaneous T-cell lymphoma (CTCL) or peripheral T cell lymphoma (PTCL).

In certain exemplary embodiments, the subject in need thereof comprises malignancies such as Mycosis Fungoides and Sezary Syndrome, and KIR3DL2-expressing autoimmune disorders.

### DETAILED DESCRIPTION

Before the disclosure is described, it is to be understood that the disclosure is not limited to methods and experimental conditions described herein, as such methods and conditions may vary. It is also to be understood that the terminology used herein is for the purpose of describing embodiments only, and is not intended to be limiting, because the scope of the disclosure will be limited only by the appended claims. It is also understood that the disclosure herein suitably may be practiced in the absence of any element which is not specifically disclosed herein.

Unless defined otherwise, all technical and scientific terms used herein have the same meaning as commonly understood by one of ordinary skill in the art to which this disclosure belongs.

The present disclosure may suitably comprise, consist of, or consist essentially of, the elements described herein.

Biotherapeutics (e.g., antibodies) encounter various type of stresses such as shear or interfacial stress during the development, manufacture, and clinical use of the biotherapeutics. Maintaining the integrity of these biotherapeutics is important because it affects not only the efficacy of the biotherapeutic but also the safety for patients. For example, when an antibody formulation is mixed in a vial or transferred from a vial into a syringe or another container (e.g. intravenous bag), the formulation is exposed to an air/liquid interface or exposed to different material surfaces. These interfaces can affect the biotherapeutic's quality, by forming visible particles, subvisible particles, aggregates, or change the target protein concentration due to adsorption of the molecules to various interfaces. For example, infusion solutions containing certain levels of protein aggregates or particles are not suitable for IV administration because they may reduce potency or increase immunogenicity. A biotherapeutic (e.g. an antibody) formulation that also provides for a long shelf life can help reduce costs of repeated production.

Since antibodies are so varied, no universal formulation or conditions are known for desired attributes such as long-term storage and stress stabilities. Therefore, formulations or conditions must be tailored for that antibody and desired attributes. Disclosed are formulations that may be used for biotherapeutics such as antibodies to provide stability during long term storage, transfer, in-use, or any of the reasons discussed here.

The disclosed formulations for an antibody include a buffer(s), tonicity agent(s), and surfactant(s). In one embodiment, the pharmaceutical formulation is an aqueous formulation, viz., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the antibody formulation comprises: (i) dibasic sodium phosphate; (ii) monobasic sodium phosphate; (iii) sodium chloride; and (iv) polysorbate 80. In some embodiments, the antibody formulation consists of: (i) dibasic sodium phosphate; (ii) monobasic sodium phosphate; (iii) sodium chloride; and (iv) polysorbate 80.

Stability of a pharmaceutical product may be defined as the capability of a particular formulation in a specific container/closure system to remain within its physical, chemical, and informational specifications. In other words, it is the extent to which a product retains throughout its period of storage and use the same properties and characteristics, within the specified limits, possessed at the time of its packaging. Stability testing thus evaluates the effect of environmental factors on the quality of the biotherapeutic or a formulated product which is used to predict its shelf life, determine proper storage conditions, and suggest labeling instructions.

As used herein, the term “stability” generally is related to maintaining the integrity or to minimizing the degradation, denaturation, aggregation or unfolding of the biotherapeutic such as a protein, peptide, or another bioactive macromolecule (e.g. antibody). As used herein, “improved stability” generally means that, under conditions known to result in degradation, denaturation, aggregation or particle formation, the protein (e.g., antibody such as anti-KIR3DL2 antibody), peptide or another bioactive macromolecule of interest maintains greater stability compared to a control protein, peptide, or another bioactive macromolecule.

Applicants have found that the antibody in the described formulation results in greatly reduced particle formation as determined by visual inspection, micro-flow imaging (MFI), or size-exclusion chromatography (SEC). In some embodiments, the formulation is essentially free of particles upon storage at about 2° C. to 8° C., 20° C. to 30° C. or 25° C. to 45° C. In some embodiments, the formulation is essentially free of particles upon storage at about 2° C. to 8° C., 20° C. to 30° C. or 25° C. to 45° C. for about at least 6 months, at least 12 months, 18 months, 24 month, 36 months or at least 48 months as determined by visual inspection, MFI, or SEC. In some embodiments, the formulation is essentially free from particles upon storage at about 2° C. to 5° C. for 6-12 months, 12-18 months, 18-24 months, 24-36 months, or 36 months 36-48 months.

In some embodiments, the antibody formulation is stable if it is essentially free of particles when a first container (e.g. vial) stored at 2-8° C. is transferred to another, second container (e.g. a syringe) as determined by visual inspection, MFI, or SEC. In some embodiments, the antibody formulation is stable if it is essentially free of particles when a first container (e.g. vial) stored at 2-8° C. for 6-12 months, 12-18 months, 18-24 months, 24-36 months, or 36 months 36-48 months is transferred to another, second container (e.g. a syringe) as determined by visual inspection, MFI, or SEC.

In some embodiments, stability refers to an antibody formulation having low to undetectable levels of particle formation. In some embodiments, the antibody formulation has less than 1000 particles/mL, less than 700 particles/mL, less than 600 particles/mL, less than 500 particles/mL, less than 400 particles/mL, less than 200 particles/mL, less than 100 particles/mL, less than 50 particles/mL, less than 20 particles/mL, less than 10 particles/mL as determined by visual inspection, MFI, or SEC, wherein the particles detected are greater than or equal to 10 micro meters in size or equal to 25 micro meters in size, after storage at about 2-8° C. for about 12 months to 48 months, e.g. 12 months, 24 months, 36 months.

In some embodiments, the antibody formulation has less than or equal to 200, 100 or 50 particles of ≥10 μm diameter per mL as assessed by micro flow imaging (MFI) after storage at around +5° C. (e.g. +5±3° C.) for at least 6, months, 12, 24, 36 or 48 months followed by being subjected to shear or interfacial stress. In some embodiments, the antibody formulation has 50-200 particles, 50-100 particles, 100-200 particles of ≥10 μm diameter per mL as assessed by micro flow imaging (MFI) after storage at around +5° C. (e.g. +5±3° C.) for at least 6, months, 12, 24, 36 or 48 months followed by being subjected to shear or interfacial stress.

In some embodiments the antibody formulation has less than or equal to 50, 20 or 10 particles of ≥25 μm diameter per mL as assessed by micro flow imaging (MFI) after storage at around +5° C. (e.g. +5±3° C.) for at least 6, 12, 24, 36, or 48 months followed by being subjected to shear or interfacial stress. In some embodiments, the antibody formulation has 50-200 particles, 50-100 particles, 100-200 particles of ≥25 μm diameter per mL as assessed by micro flow imaging (MFI) after storage at around +5° C. (e.g. +5±3° C.) for at least 6, months, 12, 24, 36 or 48 months followed by being subjected to shear or interfacial stress.

In some embodiments, the antibody formulation is considered stable when a first container (e.g. vial) stored at 2-8° C. for about 12 months to 48 months is transferred to another, second container (e.g. a syringe) and contains less than less than 1000 particles/mL, less than 700 particles/mL, less than 650 particles/mL, less than 500 particles/mL, less than 400 particles/mL, less than 200 particles/mL, less than 100 particles/mL, less than 50 particles/mL, less than 20 particles/mL, less than 10 particles/mL as determined by visual inspection, MFI, or SEC, wherein the particles detected are greater than or equal to 10 micro meters in size or equal to 25 micro meters in size.

In some embodiments the antibody is stable if has less than 20%, less than 15%, less than 10%, less than 5% or less than 2% of the antibody is degraded, denatured, aggregated, or unfolded as determined by HP-SEC when the antibody is stored at 2° C. to 8° C. for 6 months, 12, months, 18 months, 24 months, 36 months, or 48 months. In some embodiments the antibody is stable when a first container (e.g. vial) after storage at 2-8° C. is transferred to another, second container (e.g. a syringe) and it has less than 20%, less than 15%, less than 10%, less than 5% or less than 2% of the antibody is degraded, denatured, aggregated, or unfolded as determined by HP-SEC when the antibody is stored at 2° C. to 8° C. for 6 months, 12, months, 18 months, 24 months, 36 months or 48 months.

In some embodiments, the antibody formulation is stable when it has reduced fragmentation of the antibody. In some embodiments, the antibody fragmentation levels refers to the formulation containing equal to or more than 80%, 85%, 90%, 95%, 98% or 99% of the total protein, for example, in a single peak as determined by SEC, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an antibody.

### Buffers

The disclosed formulation includes a buffer(s). In some embodiments, the buffer is a citrate, phosphate, succinate, histidine, tartrate, and maleate buffers. In some embodiments, the buffers include sodium acetate, sodium carbonate, citrate, glycylglycine, histidine, glycine, lysine, arginine, sodium dihydrogen phosphate, disodium hydrogen phosphate, sodium phosphate, sodium citrate, sodium borate, tris(hydroxymethyl)-aminomethane, bicine, tricine, malic acid, succinate, maleic acid, fumaric acid, tartaric acid, aspartic acid, or any combination thereof.

In some embodiments, the buffer, is a sodium phosphate buffer. In some embodiments the sodium phosphate buffer is a dibasic sodium phosphate and monobasic sodium phosphate buffer. In some embodiments, the sodium phosphate buffer is a dibasic sodium phosphate and monobasic sodium phosphate buffer that provides the liquid formulation with a pH close to physiological blood pH. Attaining a pH close to the physiological blood pH reduces the risk of pain or side effects on injection. In some embodiments, the only buffer is a dibasic sodium phosphate and monobasic sodium phosphate.

In some embodiments, the buffer concentration can range from about 0.1 millimolar (mM) to about 100 mM. In some embodiments, the buffer concentration is from about 0.5 mM to about 50 mM, about 1 mM to about 30 mM, about 1 mM to about 18 mM, 1 mM to about 15 mM or 1 mM to about 10 mM. In some embodiments, the buffer concentration is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM or about 50 mM. In some embodiments, the buffer concentration is 10 mM to 12 mM. In some embodiments, the buffer is about 5 mM to 12 mM of a phosphate buffer. In other embodiments, the buffer concentration is about 5 mM to 12 mM of a sodium phosphate buffer. In some embodiments, the buffer concentration is about 5 mM to 12 mM of a dibasic sodium phosphate and 1-5 mM monobasic sodium phosphate buffer.

In some embodiments, the formulation has a pH from 4 and 8, between 5 and 7.5, or between 6 and 7.5, or between or between 6.9 and 7.9, between 6.9 and 7.2, between 6.9 and 7.3, between 6.9 and 7.4 or between 6.9 and 7.5. In some embodiments, the pH of the antibody formulation is 6.9, 7.0, 7.1, 7.2, 7.3, 7.4, 7.5, 7.6, 7.8, or 7.9. In some embodiments, the pH of the antibody formulation is 7.2.

### Tonicity Agent

The disclosed formulation further includes a tonicity agent. In some embodiments, the tonicity agent is a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or any combination thereof. In some embodiments, the tonicity agent ranges from about 1 mg/ml to about 20 mg/ml.

In some embodiments, the sugar is a mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used.

In some embodiments, the tonicity agent is a salt. In some embodiments, the salt is a pharmaceutically acceptable and includes sodium chloride, sodium succinate, sodium sulfate, potassium chloride, magnesium chloride, magnesium sulfate, and calcium chloride. In some embodiments, the salt is sodium chloride. In some embodiments, the only tonicity agent is salt.

In some embodiments, the salt in the liquid formulation is from a range of concentrations of 100 mM to 250 mM, 120 mM to 250 mM, 150 to 250 mM, 200 mM to 250 mM, 100 mM-150 mM. In some embodiments, the salt concentration is about 130 mM, 132 mM, 133 mM, 134 mM, 135 mM, 136 mM, 137 mM, 138 mM, 139 mM, or 140 mM.

### Non-Ionic Surfactants

In some embodiments, the formulation includes a surfactant. The surfactant may, for example, be selected from a detergent, polyoxypropylene-polyoxyethylene block polymers (e.g. poloxamers such as poloxamer 188 and 407), polyoxyethylene sorbitan fatty acid esters, polyoxyethylene and polyethylene derivatives such as alkylated and alkoxylated derivatives (tweens, e.g. Tween-20, Tween-80 and Brij-35), and monoglycerides or ethoxylated derivatives thereof.

In some embodiments, the surfactant is a polysorbate 20, polysorbate 21, polysorbate 40, polysorbate 60, polysorbate 61, polysorbate 65, polysorbate 80, polysorbate 81, polysorbate 85, or any combination thereof. In some embodiments, the surfactant is polysorbate 80. In some embodiments, the only surfactant is a polysorbate 80.

In some embodiments, the surfactant concentration ranges from 0.01 mg/ml to 5 mg/ml, from 0.01 mg/ml to 2.0 mg/ml, from 0.01 mg/ml to 1.5 mg/ml, from 0.01 mg/ml to about 1.0 mg/ml, from 0.01 mg/ml to 0.5 mg/ml, from about 0.01 mg/ml to about 0.4 mg/ml, from about 0.01 mg/ml to about 0.3 mg/ml, from about 0.01 mg/ml to about 0.2 mg/ml, from about 0.01 mg/ml to about 0.1 mg/ml, or from about 0.01 mg/ml, to about 0.05 mg/ml. In some embodiments, the surfactant concentration is about 0.01 mg/ml, 0.02 mg/ml, about 0.05 mg/ml, about 0.07 mg/ml, about 0.08 mg/ml about 0.09 mg/ml about 0.1 mg/ml, or about 0.2 mg/ml. In some embodiments, the concentration of the surfactant is about 0.02 mg/ml, about 0.05 mg/ml, about 0.1 mg/ml, or about 0.2 mg/ml. In some embodiments, the surfactant concentration is about 0.05 to about 0.2 mg/ml as concentration in this range permits maintenance of the stability of the antibody formulation in solution by reducing the particulate formation during handling of the formulation related to mixing, transfer, and agitation. In some embodiments, the surfactant is polysorbate 80 from 0.01 to about 0.2 mg/ml for an antibody formulation having 5 mg/ml to 20 mg/ml of antibody. In some embodiments, the surfactant is polysorbate 80 at 0.1 mg/ml for an antibody formulation having 5 mg/ml to 20 mg/ml of antibody. In some embodiments, the ratio of the antibody concentration to non-ionic surfactant is about 20:0.1 to 40:0.4 (w/w). In some embodiments, the ratio of the antibody concentration to non-ionic surfactant is 30:0.2 (w/w). In some embodiments, the ratio of the antibody concentration to non-ionic surfactant is about 20:0.1 to 40:0.4 (w/w) for an antibody formulation having 5 mg/ml to 20 mg/ml of antibody. In some embodiments, the ratio of the antibody concentration to non-ionic surfactant is about 20:0.1 to 40:0.4 (w/w) for an antibody formulation having 15 mg/ml of antibody.

### Antibody

The anti-KIR3DL2 antibody and variants thereof is a cytotoxic antibody that targets the KIR3DL2 receptor on the surface of tumor cells. By design, anti-KIR3DL2 binds to and selectively eliminates KIR3DL2-expressing tumor cells via its Fc effector function. Once bound to tumor cells, anti-KIR3DL2 can mediate antigen-specific antibody-dependent cell-cytotoxicity (ADCC) and phagocytosis (ADCP) of KIR3DL2-expressing cells via the recruitment of human immune effector cells such as NK cells and macrophages.

Anti-KIR3DL2 is a humanized IgG1 monoclonal antibody (mAb) produced via a recombinant technology in Chinese Hamster Ovary (CHO) cells. The protein structure is a polypeptide containing 2 heavy chains each of 448 residues and 2 light chains each of 214 residues, connected by disulfide bridges. A total of sixteen disulfide bridges are formed in the intact protein.

The Fc fragment of the antibody structure comprises two single point mutations (S239D and I332E, Kabat numbering) located in the CH2 domain of each heavy chain. These mutations were introduced to enhance the affinity of the molecule for activating Fc-receptors expressed by immune effector cells.

The intracellular post-translational processing of anti-KIR3DL2 comprises glycosylation of two N-glycosylation sites (asparagine-297 of both heavy chains) with predominantly a presence of fucosylated N-linked bi-antennary glycans. GOF is the most abundant form of N-glycans. The N-terminal of the heavy chain is blocked by a pyro-glutamic acid and the main molecular form of the antibody does not contain the heavy chain C-terminal lysine (K448) but ends at G447.

The amino acid sequence of the anti-KIR3DL2 heavy and light chains is shown below. The variable regions are highlighted in grey, N-glycosylation site is highlighted in bold, and Kabat CDRs are underlined.

Heavy chain
(SEQ ID NO: 1)
QIQLVQSGSE LKKPGASVK SCKASGYTF TAGMQWVRQ PGQGLEWIG
INSHSGVPKY AEDFKGRFVF SLDTSVSTAY LQISSLKAED TAVYFCARGG
DEGVMDYWG GTTVTVSSAS TKGPSVFPLA PSSKSTSGGT AALGCLVKDY
FPEPVTVSWN SGALTSGVHT FPAVLQSSGL YSLSSVVTVP SSSLGTQTYI
CNVNHKPSNT KVDKRVEPKS CDKTHTCPPC PAPELLGGPD VFLFPPKPKD
TLMISRTPEV TCVVVDVSHE DPEVKFNWY DGVEVHNAKT KPREEQYNST
YRVVSVLTVL HQDWLNGKE KCKVSNKALP APEEKTISKA KGQPREPQVY
TLPPSREEMT KNQVSLTCLV KGFYPSDIAV EWESNGQPEN NYKTTPPVLD
SDGSFFLYSK LTVDKSRWQ GNVFSCSVM EALHNHYTQK SLSLSPGK
Light Chain
(SEQ ID NO: 2)
DIQMTQSPSF LSASVGDRVT ITCKASQDVS TAVAWYQQKP GQPPKLLIYW
TSTRHTGVPD RFSGSGSGTD YTLTISSLQA EDVAVYYCQQ HYSTPWTFGG
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC

The molecular mass of the individual anti-KIR3DL2 molecules (in different forms) depends on the pattern of actual glycosylation and other post-translational modifications. The average molecular weight of the anti-KIR3DL2 forms determined by MALDI-TOF mass spectrometry is approximately 148 kDa. The molecular mass of the most dominant forms of anti-KIR3DL2 antibody has been confirmed by high resolution mass spectrometry.

In some embodiments, the anti-KIR3DL2 antibody is referred to as IPH4102 with a CAS NR 2187368-16-5 and with an international non-proprietary name as Lacutamab. In some embodiments, the antibody has the sequence of the heavy chain of SEQ ID NO: 1 and light chain of SEQ ID NO: 2. In some embodiments, the antibody does not contain the heavy chain C-terminal lysine (K448).

The anti-KIR3DL2 has several isoforms with isoelectric points in the pH interval 7.5 to 8.5. The estimated extinction coefficient of IPH4102 at 280 nm is 1.49 mg-1·mL·cm-1.

In some embodiments, the anti-KIR3DL2 antibody comprises the heavy and light chains variable domains disclosed in PCT international application publication no. WO 2015/136052 incorporated herein by reference in its entirety.

In some embodiments, the amino acid sequences for the light and heavy chain variable regions for anti-KIR3DL2 antibodies referred to as 2B12 are shown below (L indicates light chain, H indicates heavy chain).

2B12-L :
(SEQ ID NO: 3)
DIQMTQSPSFLSASVGDRVTITCKASQDVSTAVAWYQQKPGQPPK
LLIYWTSTRHTGVPDRFSGSGSGTDYTLTISSLQAEDVAVYYCQQ
HYSTPWTFGGGTKVEIK
2B12-L2:
(SEQ ID NO: 4)
DIVMTQSPSFLSASVGDRVTITCKASQDVSTAVAWYQQKPGQPPK
LLIYWTSTRHTGVPDRFSGSGSGTDYTLTISSVQAEDVAVYYCQQ
HYSTPWTFGGGTKVEIK
2B12-H2:
(SEQ ID NO: 5)
QIQLVQSGSELKKPGASVKVSCKASGYTFTTAGMQWVRQAPGQGL
EWIGWINSHSGVPKYAEDFKGRFVFSLDTSVSTAYLQISSLKAED
TAVYFCARGGDEGVMDYWGQGTTVTVSS

In some embodiments, the CDR sequences of the anti-KIR3DL2 antibody (e.g. 2B12) are as follows: a HCDR1 region comprising an amino acid sequence TAGMQ (SEQ ID NO: 6) or a sequence of at least 3 or 4 contiguous amino acids thereof; a HCDR2 region comprising an amino acid sequence WINSHSGVPKYAEDFK (SEQ ID NO: 7) or a sequence of at least 4, 5, 6, 7, 8, 9 or 10 contiguous amino acids thereof; a HCDR3 region comprising an amino acid sequence GGDEGVMDY (SEQ ID NO: 8) or a sequence of at least, 5, 6, 7, or 8 contiguous amino acids thereof; a LCDR1 region comprising an amino acid sequence KASQDVSTAVA (SEQ ID NO: 9) or a sequence of at least 4, 5, 6, 7, 8, 9 or 10 contiguous amino acids thereof; a LCDR2 region comprising an amino acid sequence WTSTRHT (SEQ ID NO: 10) or a sequence of at least 3, 4 or 5 contiguous amino acids thereof; or a LCDR3 region comprising an amino acid sequence QQHYSTPWT (SEQ ID NO: 11) or a sequence of at least 4, 5, 6, 7, or 8 contiguous amino acids thereof.

In some embodiments, the antibody in the formulation is from 1 mg/ml to 500 mg/ml, from 10 mg/ml to 500 mg/ml, from 20 mg/ml to 400 mg/ml, from 30 mg/ml to 300 mg/ml, from 40 mg/ml to 400 mg/ml. In some embodiments, the antibody is present at a concentration from 0.5 to 20 mg/ml, 1 to 15 mg/ml, 1 to 15 mg/ml, 5 to 10 mg/ml, 10 mg/ml to 15 mg/ml, 10 mg/ml to 30 mg/ml, 10 mg/ml to 20 mg/ml, or 12 mg/ml to 18 mg/ml.

In some embodiments, the antibody concentration is at least about 1 mg/ml, about 5 mg/ml, about 10 mg/ml, about 15 mg/ml, or about 20 mg/ml. In some embodiments, the anti-KIR3DL2 antibody and variants thereof (e.g. Lacutamab) in the formulation is from 1 mg/ml to 500 mg/ml, from 10 mg/ml to 500 mg/ml, from 20 mg/ml to 400 mg/ml, from 30 mg/ml to 300 mg/ml, from 40 mg/ml to 400 mg/ml. In some embodiments, the KIR3DL2 antibody and variants thereof (e.g. Lacutamab) antibody concentration is from about 1 mg/ml, about 5 mg/ml, about 10 mg/ml, about 15 mg/ml, or about 20 mg/ml.

In some embodiments, the antibody formulation has a pH from 4 and 8, between 5 and 7.5, between 6 and 7.5, between 6 and 7.3, between 6.9 and 7.9, between 6.9 and 7.5, between 6.9 and 7.2, between 6.9 and 7.3, or between 7.1 and 7.3. In some embodiments, the pH of the antibody formulation is 6.9, 7.0, 7.1, 7.2, 7.3, 7.4, 7.5, 7.6, 7.8, or 7.9. In some embodiments, the pH of the antibody formulation is 7.2.

In some embodiments, the antibody formulation has an antibody at 15 mg/ml, buffer at 10.52 mg/ml to 1.151 mg/ml, tonicity agent at 137 mM, surfactant at 0.1 mg·ml and pH of 7.2. In some embodiments, the antibody formulation has anti-KIR3DL2 antibody at 15 mg/ml, disodium phosphate dihydrate at 1.51 mg/ml, sodium dihydrogen phosphate dihydrate at 0.52 mg/ml, sodium chloride salt at 137 mM, polysorbate 80 at 0.1 mg·ml and pH of 7.2.

In some embodiments, the antibody formulation stored for at least 6 months to 48 months is prepared for use by pooling the drug, viz., transferring the stored antibody formulation into another container (e.g. a syringe). During the transfer, a non-ionic surfactant is mixed with the stored antibody to provide a stabilized antibody formulation comprising a ratio of antibody to non-ionic surfactant of 30:0.2 (w/w). In some embodiments, the ratio of the antibody concentration to non-ionic surfactant is about 20:0.1 to 40:0.4 (w/w).

Various types of syringes can be used. In some embodiments, the syringe is a plastic syringe or a glass syringe.

In some embodiments, the syringe is a pre-filled with the antibody formulation, the pre-fille syringe stored for 6 months to 48 months. In some embodiments, the syringe is pre-filled, the pre-filled syringe stored for greater than 6 months, 12 months, 18 months, 24 months, 36 months, or 48 months prior to administration to a subject.

In some embodiments, the pre-filled syringe comprises (a) about 5-20 mg/ml mg/mL of the antibody, buffer at 0.52 mg/ml to 1.51 mg/ml, tonicity agent at 137 mM, surfactant at 0.01 mg·ml and pH of 7.2.

The antibody formulation may be administrated by various routes. In some embodiments, the administration route is subcutaneous, intramuscular, intraperitoneal, intravenous, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment. In some embodiments, the administration route of the antibody formulation is intravenous.

Some embodiments are directed to a kit comprising any of the antibody formulations described herein, the containers described herein (e.g. one, two, three or more vials), the dosage forms described herein, or the pre-filled syringe described herein.

In some embodiments, the formulation is a stable aqueous pharmaceutical formulation comprising of, consisting essentially of, or consists of:
(i) an anti-KIR3DL2 antibody that binds to a KIR3DL2 receptor;
(ii) one or more buffers;
(iii) a tonicity agent; and
(iv) a non-ionic surfactant;
wherein the ratio of the antibody concentration to non-ionic surfactant is about 20:0.1 to 40:0.4.

In some embodiments, the formulation is a stable aqueous pharmaceutical formulation comprising of, consisting essentially of, or consists of:
(i) an anti-KIR3DL2 antibody that binds to a KIR3DL2 receptor;
(ii) dibasic sodium phosphate;
(iii) monobasic sodium phosphate; and
(iv) sodium chloride; wherein the ratio of the antibody concentration to non-ionic surfactant is about 20:0.1 to 40:0.4.

In some embodiments the antibody formulation comprises, consists essentially of, or consists of (i) an anti-KIR3DL2 antibody that binds to a KIR3DL2 receptor at a concentration of at least 15 mg/ml; and (ii) polysorbate 80 at a concentration of 0.02-0.2 mg/ml; wherein the formulation further comprises: (a) one or more buffers; and (b) a tonicity agent.

In some embodiments the antibody formulation comprises, consists essentially of, or consists of: (i) an anti-KIR3DL2 antibody that binds to a KIR3DL2 receptor at a concentration of at least 15 mg/ml; and (ii) polysorbate 80 at a concentration of 0.02-0.2 mg/ml; wherein the formulation further comprises: (a) dibasic sodium phosphate; (b) monobasic sodium phosphate; and (c) sodium chloride.

Some embodiments are directed to a method of producing a stable antibody formulation comprising an antibody, the method comprising: (a) purifying an antibody or antigen binding fragment thereof to about 5 mg/ml to 20 mg/ml, wherein the antibody is an anti-KIR3DL2 antibody; and (b) placing the isolated antibody in a stabilizing formulation to form the stable antibody formulation, wherein the resulting stable antibody formulation comprises: buffer at 0.52 mg/ml to 1.51 mg/ml, tonicity agent at 137 mM, surfactant at 0.01 mg·ml and pH of 7.2. In some embodiments, is directed to a method of producing a stable antibody formulation comprising an antibody, the method comprising: (a) purifying an antibody or antigen binding fragment thereof to about 5 mg/ml to 20 mg/ml, wherein the antibody is an anti-KIR3DL2 antibody; and (b) placing the isolated antibody in a stabilizing formulation to form the stable antibody formulation, wherein the formulation is any formulation described herein.

### Method of Treatment

Provided are methods of using the antibody; for example, a method for inhibiting cell proliferation or activity, for delivering a molecule into a cell (e.g. a toxic molecule, a detectable marker, etc.), for targeting, identifying or purifying a cell, for depleting, killing or eliminating a cell, for reducing cell proliferation, the method comprising exposing a cell, such as a T cell which expresses a KIR3DL2 polypeptide, to an antigen-binding compound (e.g. anti-KIR3DL2 antibody) that binds a KIR3DL2 polypeptide. It will be appreciated that for the purposes of the present disclosure, “cell proliferation” can refer to any aspect of the growth or proliferation of cells, e.g., cell growth, cell division, or any aspect of the cell cycle. The cell may be in cell culture (in vitro) or in a mammal (in vivo), e.g. a mammal suffering from a KIR3DL2-expressing pathology. Also provided is a method for inducing the death of a cell or inhibiting the proliferation or activity of a cell which expresses a KIR3DL2 polypeptide, comprising exposing the cell to an antigen-binding compound that binds a KIR3DL2 polypeptide linked to a toxic agent, in an amount effective to induce death and/or inhibit the proliferation or activity of the cell. Thus, provided is a method for treating a mammal suffering from a proliferative disease, and any condition characterized by a pathogenic expansion or activation of cells expressing of a KIR3DL2 polypeptide, the method comprising administering a pharmaceutically effective amount of an antigen-binding compound (e.g. anti-KIR3DL2 antibody) to the mammal. In some embodiments the antibody is used to treat such conditions as Sezary Syndrome, Mycosis Fungoides, CTCL, a peripheral T cell lymphoma, an ortho visceral extranodal PTCL (e.g., an NK/T-lymphoma or an enteropathy associated T cell lymphoma (EATL)), an anaplastic large cell lymphoma (ALCL), a PTCL-NOS (Not Otherwise Specified), and autoimmune or inflammatory conditions, e.g. arthritis, ankylosing spondylitis, cardiovascular disease. In some embodiments, the antibody is used to treat a cutaneous T-cell lymphoma (CTCL) or peripheral T cell lymphoma (PTCL).

In some embodiments is a method of treating comprising: administering to a subject in need thereof an effective amount of a pharmaceutical formulation comprising: (i) an anti-KIR3DL2 antibody that binds to a KIR3DL2 receptor at a concentration of at least 15 mg/ml; and (ii) polysorbate 80 at a concentration of 0.02-0.2 mg/ml; wherein the formulation further comprises: (a) one or more buffers; and (b) a tonicity agent.

In some embodiments is a method of treating malignancies involving CD4+ T cells that express KIR3DL2 receptors or Mycosis Fungoides and Sezary Syndrome, and KIR3DL2-expressing autoimmune disorders comprising: administering to a subject in need thereof an effective amount of a pharmaceutical formulation comprising: (i) an anti-KIR3DL2 antibody that binds to a KIR3DL2 receptor at a concentration of at least 15 mg/ml; and (ii) polysorbate 80 at a concentration of 0.02-0.2 mg/ml; wherein the formulation further comprises: (a) dibasic sodium phosphate; (b) monobasic sodium phosphate; and (c) sodium chloride.

### EXAMPLES

### Example 1

A study was conducted to develop a liquid formulation for an intravenous (IV) administration of the anti-KIR3DL2 antibody (lacutamab) having the heavy and light chains sequences in SEQ ID NO: 1 and 2, respectively. The antibody concentration was formulated at 15 mg/ml based on a flat dose of 750 mg dose to be administered.

From previous development of a formulation used for aggregation propensity studies (See WO 2015/136052) a anti-KIR3DL3 antibody sharing the VH and VL domains was formulated with the following buffer: Na2HPO4=10 mM and NaH2PO4=1.8 mM at a pH of about 7.4. This pH showed the highest temperature of aggregation and corresponded to the blood's physiological pH, which would be well tolerated by a patient for IV administration.

To determine the osmolality agent with the above buffers, three osmolality agents were selected: sucrose, trehalose, and sodium chloride (NaCl). The sucrose and trehalose concentrations were selected at 220 mM and NaCl at 137 mM each in a 10 mM phosphate buffer to achieve an osmolality that would be compliant with European Pharmacopoeia monograph for monoclonal antibodies specified as ≥240 mOsm and to be close to the blood's osmolality of approximately 280˜300 mOsm. The osmolality of these aqueous solutions is shown in Table 1.

TABLE 1

| | | | Triplicates | | Mean |
|---|---|---|---|---|---|
| | | Concentrations | osmolality | SD | osmolality |
| Solutions | Products | (mM) | (mOsm) | (mOsm) | (mOsm) |
| S1 | Na₂HPO₄ | 10 | 284 | 1 | 285 |
| | NaH₂P0₄ | 1.8 | 285 | | |
| | Sucrose | 220 | 285 | | |
| S2 | Na₂HPO₄ | 10 | 282 | 3 | 284 |
| | NaH₂PO₄ | 1.8 | 284 | | |
| | Trehalose | 220 | 287 | | |
| S3 | Na₂HPO₄ | 10 | 282 | 0 | 282 |
| | NaH₂PO₄ | 1.8 | 282 | | |
| | NaC1 | 137 | 282 | | |

Next, the surfactant concentrations were determined based on the above S1, S2, and S3 solutions with a 15 mg/ml antibody concentration. Three polysorbate 80 concentrations (0.01 mg/mL, 0.1 mg/mL, and 0.5 mg/mL) were tested. Table 2 shows the nine formulations studied for their stability.

TABLE 2

| Formulations | Products | Function | Concentrations | |
|---|---|---|---|---|
| F1a | IPH4102 | Active | 15 | mg/mL |
| | Na₂HPO₄ | Buffer | 10 | mM |
| | NaH₂PO₄ | Buffer | 1.8 | mM |
| | Sucrose | Isotonifier | 220 | mM |
| | Polysorbate 80 | Surfactant | 0.01 | mg/mL |
| F1b | IPH4102 | Active | 15 | mg/mL |
| | Na₂HPO₄ | Buffer | 10 | mM |
| | NaH₂PO₄ | Buffer | 1.8 | mM |
| | Sucrose | Isotonifier | 220 | mM |
| | Polysorbate 80 | Surfactant | 0.1 | mg/ml |
| F1c | IPH4102 | Active | 15 | mg/mL |
| | Na₂HPO₄ | Buffer | 10 | mM |
| | NaH₂PO₄ | Buffer | 1.8 | mM |
| | Sucrose | Isotonifier | 220 | mM |
| | Polysorbate 80 | Surfactant | 0.5 | mg/ml |
| F2a | IPH4102 | Active | 15 | mg/ml |
| | Na₂HPO₄ | Buffer | 10 | mM |
| | NaH₂PO₄ | Buffer | 1.8 | mM |
| | Trehalose | Isotonifier | 220 | mM |
| | Polysorbate 80 | Surfactant | 0.01 | mg/ml |
| F2b | IPH4102 | Active | 15 | mg/ml |
| | Na₂HPO₄ | Buffer | 10 | mM |
| | NaH₂PO₄ | Buffer | 1.8 | mM |
| | Trehalose | Isotonifier | 220 | mM |
| | Polysorbate 80 | Surfactant | 0.1 | mg/ml |
| F2c | IPH4102 | Active | 15 | mg/ml |
| | Na₂HPO₄ | Buffer | 10 | mM |
| | NaH₂PO₄ | Buffer | 1.8 | mM |
| | Trehalose | Isotonifier | 220 | mM |
| | Polysorbate 80 | Surfactant | 0.5 | mg/ml |
| F3a | IPH4102 | Active | 15 | mg/mL |
| | Na₂HPO₄ | Buffer | 10 | mM |
| | NaH₂PO₄ | Buffer | 1.8 | mM |
| | NaCl | Isotonifier | 137 | mM |
| | Polysorbate 80 | Surfactant | 0.01 | mg/ml |
| F3b | IPH4102 | Active | 15 | mg/mL |
| | Na₂HPO₄ | Buffer | 10 | mM |
| | NaH₂PO₄ | Buffer | 1.8 | mM |
| | NaCl | Isotonifier | 137 | mM |
| | Polysorbate 80 | Surfactant | 0.1 | mg/ml |
| F3c | IPH4102 | Active | 15 | mg/mL |
| | Na₂HPO₄ | Buffer | 10 | mM |
| | NaH₂PO₄ | Buffer | 1.8 | mM |
| | NaCl | Isotonifier | 137 | mM |
| | Polysorbate 80 | Surfactant | 0.5 | mg/ml |

Three temperature storage conditions were used to identify the most stable formulation after eight weeks (T8W): 5° C.±3° C., (long term storage) 25° C.±2° C. (accelerated storage) and 40° C.±2° C. (stress storage) and up to 6 months at 5° C.±3° C. and 25° C.±2° C.

The stability of these formulations was also monitored following three freeze/thaw (F/T) cycles (<−65° C./room temperature) and a shaking stress (3000 rpm for 3 minutes). Table 3 shows the thermal stability plan.

TABLE 3

| Storage | | | | | | | |
|---|---|---|---|---|---|---|---|
| Temperature & | Time paint& (W = weeks. M = Months) | | | | | | |
| Stress conditions | TO | T2W | T4W | T6W | TSW | T3M* | T6M* |
| +5° C. | X | X | X | X | X | X | X |
| +25° C. | | X | X | X | X | X | X |
| +40° C. | | X | X | X | X | | |
| 3 F/T | | X | | | | | |
| Shaking | | X | | | | | |

*For the most stable formulation selected after T8W only

Stability under the various conditions (freeze/thaw, shaking stress, and thermal stress) were evaluated for the following:
1. Appearance
2. Sub-visible particles count by Micro Flow Imaging (MFI)
3. pH
4. Osmolality
5. Protein Concentration
6. SE-HPLC
7. SDS-PAGER
8. SDS-PAGE NR
9. cIEF
10. Bioassay

### Freeze Thaw Results

The freeze/thaw (F/T) study consisted in 3 F/T cycles (<−65° C./Room temperature). F/T cycles were initiated after the TO of the stability study and completed after one month. The F/T samples were analysed in line after the last thawing.

### Appearance (Visual Inspection)

TABLE 4

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Appearance | Before | CCL | CCL | CCL | CCL | CCL | CCL | CCL | CCL | CCL |
| | freeze/ | FVP | EFVP | EFVP | FVP | EFVP | FVP | EFVP | EFVP | EFVP |
| | thaw | | | | | | | | | |
| | After | CCL | CCL | CCL | CCL | CCL | CCL | CCL | CCL | CCL |
| | freeze/ | FVP | FVP | FVP | FVP | FVP | FVP | FVP | FVP | FVP |
| | thaw | | | | | | | | | |

C = Clear CL = Colorless EFVP = Essentially free of visible particles FVP = Free of visible particles.

After freeze/thaw stress, no evolution in colour (colourless), clarity (clear) or visible particles (free or essentially free of visible particles) were reported in the vials. The freeze/thaw stress did not affect the appearance of the solution and no discrimination could be made between the formulation conditions assessed.

### Sub-Visible Particle Cout (MFD)

Table 5 shows the results of sub-visible particles as particles/mL and as determined by MFI.

TABLE 5

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Particles >10 μm | Before | 1334 | 528 | 48 | 66 | 72 | 41 | 28 | 87 | 45 |
| | freeze/ | | | | | | | | | |
| | thaw | | | | | | | | | |
| | After | 47 | 1758 | 756 | 82 | 87 | 192 | 20 | 114 | 46 |
| | freeze/ | | | | | | | | | |
| | thaw | | | | | | | | | |
| Particles >25 μm | Before | 201 | 99 | 11 | 19 | 6 | 9 | 9 | 12 | 10 |
| | freeze/ | | | | | | | | | |
| | thaw | | | | | | | | | |
| | After | 10 | 424 | 101 | 13 | 21 | 39 | 3 | 16 | 7 |
| | freeze/ | | | | | | | | | |
| | thaw | | | | | | | | | |

Sub-visible particles evaluated by MFI showed that the sucrose-containing formulation had higher particle levels for both particle sizes monitored. In general, freezing led to an increase of sub-visible particles in the formulation containing trehalose, whereas the formulation containing NaCl appeared to stabilize the product after 3 F/T cycles as no significant increase of sub-visible particles were reported. No clear trend was observed regarding PS80 concentrations. It appeared that the PS80 amount did not affect the stability of the product after 3 F/T cycles.

### Impurities (SE-HPLC)—

Table 6 shows the impurities measured by size exclusion-high performance liquid chromatography (SE-HPLC).

TABLE 6

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Monomer | Before | 99.6 | 99.6 | 99.6 | 99.5 | 99.6 | 99.6 | 99.6 | 99.6 | 99.6 |
| (%) | freeze/ | | | | | | | | | |
| | thaw | | | | | | | | | |
| | After | 98.2 | 98.1 | 98.0 | 98.1 | 98.4 | 97.9 | 99.2 | 99.1 | 99.1 |
| | freeze/ | | | | | | | | | |
| | thaw | | | | | | | | | |
| HMWP | Before | 0.4 | 0.4 | 0.4 | 0.5 | 0.5 | 0.4 | 0.4 | 0.4 | 0.4 |
| (%) | freeze/ | | | | | | | | | |
| | thaw | | | | | | | | | |
| | After | 0.4 | 0.4 | 0.4 | 0.4 | 0.4 | 0.4 | 0.8 | 0.9 | 0.9 |
| | freeze/ | | | | | | | | | |
| | thaw | | | | | | | | | |
| LMWP | Before | 0.0 | 0.0 | 0.0 | 0.0 | 0.0 | 0.0 | 0.0 | 0.0 | 0.0 |
| (%) | freeze/ | | | | | | | | | |
| | thaw | | | | | | | | | |
| | After | 1.4 | 1.5 | 1.6 | 1.5 | 1.2 | 1.7 | 0.0 | 0.0 | 0.0 |
| | freeze/ | | | | | | | | | |
| | thaw | | | | | | | | | |

The formulation containing NaCl showed higher main peak level after F/T stress and no fragmentation phenomenon (low molecular weight protein (LMWP)) was reported. However, a slight but acceptable (less than 1% in the solution) increase of high molecular weight protein (HMWP) was reported after F/T stress for this formulation.

Finally, no effect of PS80 concentration on the generation of HMWP was reported after F/T stress.

pH

TABLE 7

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| pH value | Before | 7.4 | 7.4 | 7.4 | 7.4 | 7.4 | 7.4 | 7.2 | 7.2 | 7.2 |
| | freeze/ | | | | | | | | | |
| | thaw | | | | | | | | | |
| | After | 7.2 | 7.2 | 7.2 | 7.2 | 7.2 | 7.2 | 7.1 | 7.1 | 7.2 |
| | freeze/ | | | | | | | | | |
| | thaw | | | | | | | | | |

A slight decrease of pH was observed for formulations containing both sucrose and trehalose. However, the changes were in an acceptable range (+/−0.2) usually defined for GMP stability study. The F/T stress did not affect the pH of the NaCl-containing formulation. See Table 7 above.

Regarding impurity results, NaCl agents seemed to favour the drug product stability after F/T stress as lower HMWP and LMWP levels have been reported.

No change in protein concentration was observed during the freeze/thaw cycles. Similarly, purity results showed stable purity profiles as heavy and light chains did not decrease after freeze/thaw stress. The charge variant analysis monitored by the cIEF showed that all samples were comparable to the product reference. Potency results did not show any evolution (results are within method variability) after F/T stress for all formulation conditions evaluated. The 3 F/T cycles did not affect the potency of the drug product.

### Shaking Stress

Shaking stress consisted of vortexing a solution for 3 minutes at a speed of 3000 rpm (round per minutes). After vortexing, the solutions were stored at +5° C. for 24 hours and analysed. The results of the shaking stress are shown below at Table 8.

TABLE 8

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Appearance | Before | CCL | CCL | CCL | CCL | CCL | CCL | CCL | CCL | CCL |
| | shaking | FVP | EFVP | EFVP | FVP | EFVP | FVP | EFVP | EFVP | EFVP |
| | (T0) | | | | | | | | | |
| | After | CCL | CCL | CCL | CCL | CCL | CCL | CCL | CCL | CCL |
| | shaking | FVP | FVP | EFVP | EFVP | FVP | FVP | FVP | FVP | EFVP |

C = Clear CL = Colorless EFVP = Essentially free of visible particles FVP = Free of visible particles.

After shaking stress, no changes were observed in color (colorless), clarity (clear) or visible particles (free or essentially free of visible particles. The shaking stress did not affect the solution appearance and did not generate visible particles.

### Sub-Visible Particle Count (MFI)

TABLE 9

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Particles >10 μm | Before | 33 | 110 | 130 | 130 | 41 | 48 | 2017 | 46 | 56 |
| | shaking | | | | | | | | | |
| | After | 57 | 369 | 408 | 199 | 103 | 48 | 27 | 97 | 30 |
| | shaking | | | | | | | | | |
| Particles >2 μm | Before | 2 | 35 | 28 | 27 | 11 | 15 | 579 | 13 | 12 |
| | shaking | | | | | | | | | |
| | After | 7 | 68 | 43 | 25 | 15 | 12 | 5 | 10 | 9 |
| | shaking | | | | | | | | | |

MFI results showed a similar trend for both the sub-visible particle sizes monitored. Formulations containing sucrose and trehalose showed higher particle levels than the formulation containing NaCl. No clear conclusion could be drawn for the PS80 concentration regarding reducing sub-visible particles during shaking stress.

### SE-HPLC

TABLE 10

| | Time | Formulations | | | | | | | | |
|---|---|---|---|---|---|---|---|---|---|---|
| Parameters | point | F1a | F1b | F1c | F2a | F2b | F2c | F3a | F3b | F3c |
| Monomer (%) | Before | 98.9 | 98.9 | 98.8 | 99.0 | 98.3 | 98.5 | 99.5 | 99.5 | 99.5 |
| | shaking | | | | | | | | | |
| | After | 97.6 | 97.8 | 98.0 | 97.9 | 97.6 | 97.9 | 99.6 | 99.6 | 99.6 |
| | shaking | | | | | | | | | |
| HMWP (%) | Before | 0.5 | 0.5 | 0.4 | 0.4 | 0.5 | 0.5 | 0.5 | 0.5 | 0.5 |
| | shaking | | | | | | | | | |
| | After | 0.5 | 0.5 | 0.5 | 0.5 | 0.5 | 0.5 | 0.4 | 0.5 | 0.4 |
| | shaking | | | | | | | | | |
| LMWP (%) | Before | 0.9 | 0.7 | 0.7 | 0.6 | 1.2 | 1.1 | 0.0 | 0.0 | 0.0 |
| | shaking | | | | | | | | | |
| | After | 2.0 | 1.7 | 1.5 | 1.6 | 2.0 | 1.7 | 0.0 | 0.0 | 0.0 |
| | shaking | | | | | | | | | |

SE-HPLC results showed that a main peak decreased after shaking stress for formulations containing both trehalose and sucrose (more than 1%). However, no main peak decrease was observed for formulations containing NaCl after shaking stress.

For the HMWP parameter, no change was observed after shaking stress for all formulations tested. The decrease of the main peak observed for trehalose and sucrose condition was correlated to fragmentation. Therefore, NaCl appeared to favour the product stability during shaking stress.

Other parameters were tested that did not show differences between the formulations. Data not shown. For example, purity results showed stable purity profiles as heavy and light chains did after the shaking stress. Potency results did not show any evolution (results are within method variability) after shaking stress for all formulation conditions evaluated. pH was also not affected by shaking stress.

### Thermal Stability Results

No changes in the appearance of the tested formulations were observed during the thermal stability study in long term (5° C.), accelerated (25° C.) and stress storage conditions (40° C.). In other words, no visible particles were reported at any of the thermal stress conditions. Further, polysorbate 80 did not seem to affect the generation of visible particles.

### Sub-Visible Particle Count by MFI

TABLE 11
Sub-visible particle results during the stability study at +5° C.

| Time | Acceptance | | | | | | | | | |
|---|---|---|---|---|---|---|---|---|---|---|
| Point | criteria | F1a | F1b | F1c | F2a | F2b | F2c | F3a | F3b | F3c |
| T0 | Particles ≥10 μm | 1334 | 528 | 48 | 66 | 72 | 41 | 28 | 87 | 45 |
| | Particles ≥25 μm | 201 | 99 | 11 | 19 | 6 | 9 | 9 | 12 | 10 |
| T2W | Particles ≥10 μm | 33 | 110 | 130 | 130 | 41 | 18 | 2017 | 46 | 56 |
| | Particles ≥25 μm | 2 | 35 | 28 | 27 | 11 | 15 | 279 | 13 | 12 |
| T4W | Particles ≥10 μm | 8 | 80 | 157 | 85 | 88 | 699 | 84 | 55 | 67 |
| | Particles ≥25 μm | 2 | 16 | 34 | 11 | 34 | 18 | 19 | 11 | 12 |
| T6W | Particles ≥10 μm | 99 | 1168 | 23 | 321 | 365 | 2068 | 136 | 162 | 2742 |
| | Particles ≥25 μm | 16 | 77 | 5 | 77 | 318 | 633 | 42 | 18 | 1216 |
| T8W | Particles ≥10 μm | 1113 | 244 | 81 | 188 | 48 | 58 | 37 | 96 | 58 |
| | Particles ≥25 μm | 24 | 44 | 26 | 36 | 8 | 9 | 9 | 22 | 13 |
| T12W | Particles ≥10 μm | 384 | | | | | | 169 | | |
| | Particles ≥25 μm | 40 | | | | | | 32 | | |
| T24W | Particles ≥10 μm | 24 | | | | | | 115 | | |
| | Particles ≥25 μm | 5 | | | | | | 32 | | |

TABLE 12
Sub-visible particle results during the stability study at +25° C.

| Time | Acceptance | | | | | | | | | |
|---|---|---|---|---|---|---|---|---|---|---|
| Point | criteria | F1a | F1b | F1c | F2a | F2b | F2c | F3a | F3b | F3c |
| T0 | Particles ≥10 μm | 1334 | 528 | 48 | 66 | 72 | 41 | 28 | 87 | 45 |
| | Particles ≥25 μm | 201 | 99 | 11 | 19 | 6 | 9 | 9 | 12 | 10 |
| T2W | Particles ≥10 μm | 65 | 15 | 155 | 105 | 411 | 36 | 37 | 65 | 40 |
| | Particles ≥25 μm | 10 | 2 | 22 | 21 | 27 | 9 | 11 | 16 | 7 |
| T4W | Particles ≥10 μm | 136 | 236 | 199 | 63 | 30 | 58 | 670 | 49 | 235 |
| | Particles ≥25 μm | 22 | 55 | 44 | 8 | 6 | 16 | 197 | 10 | 11 |
| T6W | Particles ≥10 μm | 2528 | 122 | 262 | 2551 | 44 | 2491 | 29 | 83 | 59 |
| | Particles ≥25 μm | | 24 | 46 | | 10 | 11 | 2 | 4 | 7 |
| T8W | Particles ≥10 μm | | 1025 | 9205 | 2724 | 1991 | 983 | 217 | 891 | 383 |
| | Particles ≥25 μm | | 175 | 175 | 242 | 670 | 242 | 25 | 58 | 58 |

TABLE 13
Sub-visible particle results during the stability study at +40° C.

| Time | Acceptance | | | | | | | | | |
|---|---|---|---|---|---|---|---|---|---|---|
| Point | criteria | F1a | F1b | F1c | F2a | F2b | F2c | F3a | F3b | F3c |
| T0 | Particles ≥10 μm | 1334 | 528 | 48 | 66 | 72 | 41 | 28 | 87 | 45 |
| | Particles ≥25 μm | 201 | 99 | 11 | 19 | 6 | 9 | 9 | 12 | 10 |
| T2W | Particles ≥10 μm | 33 | 110 | 130 | 130 | 41 | 48 | 2017 | 46 | 56 |
| | Particles ≥25 μm | 2 | 35 | 28 | 27 | 11 | 15 | 579 | 13 | 12 |
| T4W | Particles ≥10 μm | 8 | 80 | 157 | 85 | 88 | 699 | 84 | 55 | 67 |
| | Particles ≥25 μm | 2 | 16 | 34 | 11 | 34 | 18 | 19 | 11 | 12 |
| T6W | Particles ≥10 μm | 99 | 1168 | 23 | 321 | 365 | 2068 | 136 | 162 | 2742 |
| | Particles ≥25 μm | 16 | 77 | 5 | 77 | 318 | 633 | 42 | 18 | 1216 |
| T8W | Particles ≥10 μm | 1113 | 244 | 81 | 188 | 48 | 58 | 37 | 96 | 58 |
| | Particles ≥25 μm | 24 | 44 | 26 | 36 | 8 | 9 | 9 | 22 | 13 |

Regarding MFI results, variability was observed as several formulations showed a transient increase of sub-visible particle levels (especially at +5° C. for particles >10 μm).

In the accelerated storage condition (e.g. 25° C.), particle levels for all the formulations showed an increase for both particle sizes tested. However, due to the large variability of the analysis in the samples, no discrimination between formulation conditions could be made.

In the stress storage condition (e.g. 40° C.), lower particle levels were reported, which is unexpected confirming the difficulty to highlight degradation trend and discriminate formulation conditions.

Based on these results, no degradation trend was identified concerning the generation of sub-visible particles and no discrimination between formulation conditions were made.

### Impurities

The following tables show the SE-HPLC results and the evolution of monomers and impurities (HMWP & LMWP) during the stability study in long-term accelerated and stress storage conditions.

TABLE 14
Impurities Results During Stability Study at +5° C.

| Acceptance | | | | | | | | | | |
|---|---|---|---|---|---|---|---|---|---|---|
| criteria | Time point | F1a | F1b | F1c | F2a | F2b | F2c | F3a | F3b | F3c |
| Monomer (%) | T0 | 99.6 | 99.6 | 99.6 | 99.5 | 99.6 | 99.6 | 99.6 | 99.6 | 99.6 |
| | T2W | 98.9 | 98.9 | 98.8 | 99 | 98.3 | 98.5 | 99.5 | 99.5 | 99.5 |
| | T4W | 97.7 | 97.7 | 97.9 | 38.4 | 97.8 | 98 | 99.5 | 99.5 | 99.5 |
| | T6W | 97.9 | 97.9 | 98.1 | 98.4 | 97.8 | 98 | 99.4 | 99.4 | 99.4 |
| | T8W | 97.9 | 97.9 | 98.1 | 98.8 | 98.9 | 98.9 | 99.4 | 99.4 | 99.4 |
| | T12W | 99.0 | | | | | | 99.1 | | |
| | T24W | 98.8 | | | | | | 98.9 | | |
| HMWP (%) | T0 | 0.4 | 0.4 | 0.4 | 0.5 | 0.5 | 0.4 | 0.4 | 0.4 | 0.4 |
| | T2W | 0.5 | 0.5 | 0.4 | 0.4 | 0.5 | 0.5 | 0.5 | 0.5 | 0.5 |
| | T4W | 0.5 | 0.5 | 0.5 | 0.5 | 0.5 | 0.5 | 0.5 | 0.5 | 0.5 |
| | T6W | 0.5 | 0.5 | 0.5 | 0.5 | 0.5 | 0.5 | 0.6 | 0.6 | 0.6 |
| | T8W | 0.7 | 0.7 | 0.7 | 0.6 | 0.6 | 0.8 | 0.6 | 0.6 | 0.6 |
| | T12W | 0.9 | | | | | | 0.8 | | |
| | T24W | 1.0 | | | | | | 0.9 | | |
| LMWP (%) | T0 | 0.0 | 0.0 | 0.0 | 0.0 | 0.0 | 0.0 | 0.0 | 0.0 | 0.0 |
| | T2W | 0.9 | 0.7 | 0.7 | 0.6 | 1.2 | 1.1 | 0.0 | 0.0 | 0.0 |
| | T4W | 1.8 | 1.8 | 1.6 | 1.2 | 1.8 | 1.6 | 0.0 | 0.0 | 0.0 |
| | T6W | 1.6 | 1.6 | 1.4 | 1.1 | 1.0 | 1.0 | 0.0 | 0.0 | 0.0 |
| | T8W | 0.5 | 0.5 | 0.4 | 0.6 | 0.5 | 0.4 | 0.0 | 0.0 | 0.0 |
| | T12W | .02 | | | | | | 0.1 | | |
| | T24W | 0.2 | | | | | | 0.2 | | |

TABLE 15
Impurities Results During Stability Study at +25° C.

| Acceptance | | | | | | | | | | |
|---|---|---|---|---|---|---|---|---|---|---|
| criteria | Time point | F1a | F1b | F1c | F2a | F2b | F2c | F3a | F3b | F3c |
| Monomer (%) | T0 | 99.6 | 99.6 | 99.6 | 99.5 | 99.6 | 99.6 | 99.6 | 99.6 | 99.6 |
| | T2W | 98.0 | 98.9 | 98.3 | 98.3 | 98.2 | 98.6 | 99.1 | 99.1 | 99.1 |
| | T4W | 98.2 | 97.9 | 97.8 | 98.2 | 98.1 | 98.3 | 98.5 | 98.7 | 98.7 |
| | T6W | 97.3 | 97.2 | 97.2 | 97.8 | 98 | 97.8 | 98.3 | 98.3 | 98.4 |
| | T8W | 98.2 | 98.2 | 98.4 | 98.1 | 98.2 | 97.9 | 98.6 | 98.5 | 98.3 |
| | T12W | 97.9 | | | | | | 98.2 | | |
| | T24W | 89.6 | | | | | | 91.7 | | |
| HMWP (%) | T0 | 0.4 | 0.4 | 0.4 | 0.5 | 0.5 | 0.4 | 0.4 | 0.4 | 0.4 |
| | T2W | 0.7 | 0.7 | 0.6 | 0.6 | 0.7 | 0.6 | 0.7 | 0.6 | 0.6 |
| | T4W | 0.8 | 0.8 | 0.8 | 0.7 | 0.7 | 0.7 | 0.8 | 0.7 | 0.7 |
| | T6W | 0.9 | 0.9 | 0.9 | 0.8 | 0.8 | 0.8 | 0.8 | 0.8 | 0.8 |
| | T8W | 1.2 | 1.2 | 1.4 | 1.1 | 1.1 | 1.4 | 1.0 | 1.0 | 1.2 |
| | T12W | 1.4 | | | | | | 1.1 | | |
| | T24W | 3.6 | | | | | | 1.9 | | |
| LMWP (%) | T0 | 0.0 | 0.0 | 0.0 | 0.0 | 0.0 | 0.0 | 0.0 | 0.0 | 0.0 |
| | T2W | 1.3 | 1 | 1.1 | 1.2 | 1.2 | 0.9 | 0.3 | 0.3 | 0.2 |
| | T4W | 1.0 | 1.4 | 1.5 | 1.1 | 1.0 | 1.1 | 0.7 | 0.6 | 0.6 |
| | T6W | 1.8 | 1.9 | 1.9 | 1.4 | 1.2 | 1.4 | 0.9 | 0.8 | 0.8 |
| | T8W | 0.7 | 0.7 | 0.7 | 0.8 | 0.7 | 0.7 | 0.4 | 0.5 | 0.5 |
| | T12W | 0.8 | | | | | | 0.7 | | |
| | T24W | 6.8 | | | | | | 6.4 | | |

TABLE 16
Impurities Results During Stability Study at +40° C.

| Acceptance | | | | | | | | | | |
|---|---|---|---|---|---|---|---|---|---|---|
| criteria | Time point | F1a | F1b | F1c | F2a | F2b | F2c | F3a | F3b | F3c |
| Monomer (%) | T0 | 99.5 | 99.5 | 99.5 | 99.6 | 99.6 | 99.5 | 99.6 | 99.5 | 99.5 |
| | T2W | 89.6 | 91 | 91.4 | 924 | 93.3 | 92.9 | 97.1 | 97 | 97 |
| | T4W | 87.1 | 87.8 | 88.2 | 87 | 87.6 | 89.1 | 95.2 | 94.8 | 94.3 |
| | T6W | 92.1 | 79.6 | 81.2 | 81.1 | 83.1 | 84.3 | 93.3 | 93 | 92.8 |
| | T8W | 72.7 | 71.3 | 72.1 | 69.4 | 71.8 | 73.5 | 83.6 | 82.4 | 81.9 |
| HMWP (%) | T0 | 0.5 | 0.5 | 0.5 | 0.5 | 0.5 | 0.5 | 0.5 | 0.5 | 0.5 |
| | T2W | 7.6 | 76.8 | 6.6 | 5.2 | 5.2 | 4.6 | 1.9 | 2.1 | 2.1 |
| | T4W | 9.2 | 8.7 | 8.6 | 9.8 | 8.9 | 8.1 | 3.2 | 3.5 | 4 |
| | T6W | 13.5 | 15.9 | 14.5 | 14.7 | 12.8 | 11.7 | 4.5 | 4.8 | 4.9 |
| | T8W | 16.1 | 17.4 | 16.5 | 18.9 | 16.8 | 15.4 | 5.9 | 6.3 | 6.7 |
| LMWP (%) | T0 | 0 | 0 | 0 | 0 | 0 | 0 | 0 | 0 | 0 |
| | T2W | 2.7 | 2.2 | 2 | 2.4 | 1.5 | 1.6 | 1 | 1 | 1 |
| | T4W | 3.7 | 3.6 | 3.2 | 3.2 | 3.2 | 2.9 | 1.6 | 1.7 | 1.8 |
| | T6W | 4.4 | 4.5 | 4.3 | 4.3 | 4 | 4 | 2.3 | 2.3 | 2.3 |
| | T8W | 11.2 | 11.3 | 11.4 | 11.7 | 11.5 | 11.1 | 10.5 | 11.3 | 11.4 |

Regarding soluble aggregates, the monitoring by SE-HPLC reported an increase of HMWP during the stability for all storage conditions. The stress storage condition (+40° C.) showed a significant difference between the formulation with NaCl compared to sucrose and trehalose with lower HMWP levels for NaCl. For both selected formulation conditions (F1a & F3a) after 8 weeks of stability study, the formulation condition containing NaCl seemed to limit the generation of HMWP in accelerated storage condition (+25° C.) whereas no HMWP level differences were reported in long-term storage condition (+5° C.). Moreover, the polysorbate 80 amount did not affect the generation of HWMP during the stability study for all storage conditions.

Regarding the fragments (LMWP), NaCl formulations showed less fragments than others and seemed to favor product stability.

Based on the above data evaluated under the different parameters, the observations of the study are follows and shown in Table 17:
Regarding the freeze thaw study, NaCl containing formulations showed better stability profiles based on MFI and SE-HPLC data,
Regarding the shaking stress study, formulations containing NaCl showed less degradation based on SE-HPLC data, and
Regarding the stability study (thermal stress), formulations containing NaCl showed better stability profiles based on data from SE-HPLC and SDS PAGE in reduced conditions.

Polysorbate 80 concentrations did not affect the product stability as studied during freeze thaw or shaking stress. Accordingly, the choice of osmolality agent and polysorbate 80 concentration for the liquid formulation for the anti-KIR3DL2 antibody was determined as follows:
NaCl 137 mM showed the best stability profiles over these studies.
PS80 at 0.001% (0.01 mg/ml) did not affect the product stability so the lowest concentration was selected.

TABLE 17

| | | | Freeze | | | | |
|---|---|---|---|---|---|---|---|
| | Method of | | thaw | Shaking | Stability Stady | | |
| Test | analysis | Parmeter | study | study | +5° C. | +25° C. | +40° C. |
| Appearance | Visual | Clarity | No | No | No evolution or formulation | | |
| | Inspection | Color | evolution or | evolution or | discrimination | | |
| | | Visible | formulation | formulation | | | |
| | | Particles | discrimination | discrimination | | | |
| Particulate | MFI | Ø ≥ 10 μm | Better | No | No evolution or formulation | | |
| Matter | | Ø ≥ 25 μm | stability | formulation | discrimination | | |
| | | | for | discrimination | | | |
| | | | formulations | | | | |
| | | | containing | | | | |
| | | | NaCl | | | | |
| Identity | cIEF | Comparable | Comparable | Not | Comparable | Not comparable to the | |
| | | to the | to the | performed | to the | reference | |
| | | reference | reference | | reference | | |
| Impurities | SE-HPLC | Monomer | Lower | No | No | Lower HMWP | |
| | | IgG | LMWP | decrease of | formulation | increase for | |
| | | (%Aᵣₑₐ) | increase | monomers | discrimination | formulations | |
| | | HMWP | for | for | | containing NaCl | |
| | | (%_{Area}) | formulation | formulations | | | |
| | | LMWP | containing | containing | | | |
| | | (%_{Area}) | NaCl | NaCl | | | |
| Potency | Bioassay | EC50 | No | No | Higher potency levels with | | |
| | | ratio (%) | evolution or | evolution or | formulations containing NaCl | | |
| | | | formulation | formulation | | | |
| | | | discrimination | discrimination | | | |
| Purity | SDS- | IgG as | No | No | No | No | Lower |
| | PAGE R | heavy and | evolution | evolution | formulation | formulation | Heavy |
| | | light | or | or | discrimination | discrimination | chain |
| | | chains | formulation | formulation | | | degradation |
| | | | discrimination | discrimination | | | for |
| | | | | | | | formulations |
| | | | | | | | containing |
| | | | | | | | NaCl |

| | | | | | |
|---|---|---|---|---|---|
| pH | Potentiometric | pH value | No | No | No evolution or formulation |
| | Method | | evolution or | evolution or | discrimination |

| | |
|---|---|
| formulation | formulation |
| discrimination | discrimination |

| | | | | | |
|---|---|---|---|---|---|
| Protein | Absorbance | Concentration | No | Not | No evolution or formulation |
| Concentration | at 280 mm | (mg/mL) | evolution or | performed | discrimination |

formulation
discrimination

### Example 2

At a clinical site during dose preparation of pooling the contents of several vials into a single syringe, visible particles were detected. Visible particles were not detected in the formulation before transfer from the vials to the syringe. The doses transferred were from a 34-month-old batch of the anti-KIR3DL3 antibody (lacutamab) in a formulation comprising the following:

TABLE 18

| Anti-KIR3DL2 antibody | 15 | mg/mL |
|---|---|---|
| (Drug Substance) | | |
| Na₂HPO₄, 2H₂O | 8.5 mM | |
| (Disodium phosphate | (1.51 mg/mL) | |
| dihydrate) | | |
| NaH₂PO₄, 2H₂O | 3.3 mM | |
| (Sodium dihydrogen | (0.52 mg/mL) | |
| phosphate dihydrate) | | |
| NaCl | 137 mM | |
| (Sodium chloride) | (8.01 mg/mL) | |
| Tween 80 | 0.01 | mg/mL |

| | |
|---|---|
| (Polysorbate 80) | |
| WFI | q.s. |
| (Water for Injection) | |
| pH | 7.2 |

The following example describes a formulation for reducing particle formation upon transfer from one container to another of a 36-month-old batch of the anti-KIR3DL2 antibody formulation stored at 2° C. to 8° C.

Various concentrations of PS-80 were studied by spiking the 36-month-old batch of the anti-KIR3DL2 antibody formulation in the vial. The spiked solution was then transferred from different vials into a syringe. The transferred material in the syringe was inspected for sub-visible and visible particles by visually inspecting the samples and by MFI and for impurities by SEC-HPLC.

The results are presented in Table 19 below.

TABLE 19

| | PS80 | | | | | | | | | | | | |
|---|---|---|---|---|---|---|---|---|---|---|---|---|---|
| | final | | | | | | | | | | | | |
| | content | T0 = in vials | | | | | | After pooling in syringes | | | | | |
| Test | (mg/ML) | 0.01 | 0.015 | 0.02 | 0.05 | 0.1 | 0.2 | 0.01 | 0.015 | 0.02 | 0.05 | 0.1 | 0.2 |
| Appearance | colour | CCL | CCL | CCL | CCL | CCL | CCL | CCL | CCL | CCL | CCL | CCL | CCL |
| (Visual | Visible | EFVP | EFVP | EFVP | EFVP | EFVP | EFVP | EFVP | EFVP | EFVP | EFVP | EFVP | EFVP |
| Inspection) | particles | | | | | | | | | | | | |
| Particulate | ≥10 μm | 10 | 64 | 17 | 8 | 4 | 15 | 9143 | 575 | 259 | 71 | 174 | 59 |
| Matter | particles/ | | | | | | | | | | | | |
| (MFI) | mL | | | | | | | | | | | | |
| | ≥25 μm | 2 | 2 | 2 | 0 | 2 | 0 | 917 | 29 | 46 | 14 | 35 | 4 |
| | particles/ | | | | | | | | | | | | |
| | mL | | | | | | | | | | | | |
| Impurities | Monomer | 96.7 | 96.7 | 96.7 | 96.7 | 96.7 | 96.6 | 96.8 | 96.8 | 96.7 | 96.7 | 96.7 | 96.6 |
| (SE-HPLC) | IgG | | | | | | | | | | | | |
| | Aggregates | 1.0 | 1.0 | 1.0 | 1.0 | 1.0 | 1.0 | 1.0 | 1.0 | 1.0 | 1.0 | 1.0 | 1.0 |
| | LMWP % | 2.3 | 2.3 | 2.4 | 2.3 | 2.3 | 2.4 | 2.3 | 2.3 | 2.3 | 2.3 | 2.3 | 2.3 |

CCL = clear colourless
VP = visible particles
EFVP = essentially free of visible particles

Beyond a final concentration of PS80 of 0.05 mg/mL, no visible particles were detected in the pooling syringe and the level of sub-visible particles was comparable to the level observed in vials before the transfer. Thus, a concentration of 0.1 mg/mL of PS80 was determined to reduce particle formation.

### Example 3

The anti-KIR3DL2 antibody (lacutamab) was formulated for intravenous (IV) administration as described in Example 2. Therefore, the anti-KIR3DL2 antibody had a target concentration of 10 ppm (parts per million) of polysorbate 80 (PS80). During the pooling of vials into syringes at a clinical site of a 36-month formulation that had been in storage at 2-8° C., particles were observed in the syringe. The stability of this product was therefore further evaluated because the administration device was changed to an IV bag connected to an in-line filter. The in-use stability study in bags was conducted with the antibody at that had a six-month or 36-month shelf-life. Finally, the study was also conducted to evaluate the particles that were observed during pooling.

Accordingly, the drug product with a target 10 ppm PS80 was assessed along with samples in which the antibody was spiked with a target concentration of 100 ppm of PS80. The bags were then stored for 24 hours at room temperate and ambient light. The study design is shown in Table 20.

TABLE 20

| | | Target | | | |
|---|---|---|---|---|---|
| | | Product | | | |
| Target•PS8O | | Concentration | | | |
| Concentration | | (mg/ML) | Device | Time point | Testing |
| 10 | pμm | 15.0 | PP/PE | T0; T24 h/filter | Appearance, Particulate |
| | | | bags and | | Matter, Identify, Protein |
| | | | PVC sets, | | Concentration Potency, |
| | | | PES filter | | Impurities |
| 100 | pμm | | | | Appearance, Particulate |

Matter

For unspiked samples, 10 vials were transferred one by one into a bag. The content of each vial was directly transferred into the bag with a syringe and needle. The needle was changed for each vial.

For the spiked sample with 100 ppm of PS80, 45 μL of a PS80 stock solution at 10 g/L was added to each vial to reach 100 ppm of PS80. Then the vials were crimped, and their content was withdrawn and transferred to the dedicated bag using a 10 ml syringe (each vial is transferred one by one into the bag).

### Visual Inspection (Appearance)

TABLE 21

| PS80 Concentration | | 10 ppm | | 100 ppm | |
|---|---|---|---|---|---|
| Time points | | T0 | T24 h/filtration | T0 | T24 h/filtration |
| Target | Clear to slightly | CCL | CCL | CCL | CCL |
| Value | opalescent, Colourless to | | | | |
| | slightly yellow or brown | | | | |
| | Essentially free from | VP | EFVP | EFVP | EFVP |
| | visible particles | | | | |

Visible particles were observed at TO after transfer to the bag for the samples with 10 ppm of PS80, whereas no particles were visible in the vials before transfer. Results of visual inspection is shown in Table 21.

For the spiked samples (100 ppm of PS80), no particles appeared after the transfer into the bags. An increase of PS80 concentration up to 100 ppm was found to prevent particle generation during the transfer step. At 24 hours (i.e., after filtration), no particles were observed for the unspiked and spiked samples. The filter was efficient to remove particles that were generated during the transfer to bags.

TABLE 22

| PS80 Concentration | | 10 ppm | | 100 ppm | |
|---|---|---|---|---|---|
| Time points | | T0 | T24 h/filtration | T0 | T24 h/filtration |
| Target | Particles ≥10 μm (≤6000 | 1940 | 32 | 44 | 30 |
| Value | particles/mL) | | | | |
| | Particles ≥25 μm (≤600 | 235 | 6 | 1 | 7 |
| | particles/mL) | | | | |

Table 22 shows the particle matter as determined by MFI. Particulate matter results showed a high particle level for the 10 ppm unspiked sample after transfer. This result was consistent with the visual inspection of particles during the transfer to the bag. For the spiked sample at 100 ppm, the sub visible particle levels at TO were low for both particle sizes and comparable to values reported in vials. At T24h (i.e., after filtration), both samples (unspiked vs. spiked) showed low particle levels. This indicated that the filter was efficient to remove sub visible particles. The integrity and concentration of the antibody were unaffected (results not shown).

### Example 4

Based on the results from Example 3, the anti-KIR3DL2 antibody (lacutamab) was formulated in 7 different formulation compositions (described in the following Table 23) in a stability study designed to assess the effect of variations around the formulation specifications described in Example 3. The stability study was conducted in long-term (+5±3° C.), accelerated (+25±2° C.) and stress (+40±2° C.) storage conditions, and assessed by Micro Flow Imaging (MFI), Dynamic Light Scattering (DLS), LC-MS and CE-SDS NR methodologies.

TABLE 23
Formulation compositions

| | | | Sodium | | | |
|---|---|---|---|---|---|---|
| | | | Phosphate | | | |
| Formulation | Formulation | Protein | Dibasic | NaCl | PS80 | |
| Number | Name | (mg/mL) | (mM) | (mM) | (mg/L) | pH |
| 1 | Centre Point | 16.5 | 11.8 | 137 | 100 | 7.2 |
| 2 | Low pH | | | | | 6.9 |
| 3 | High pH | | | | | 7.5 |
| 4 | Low PS80 | | | | 50 | 7.2 |
| 5 | Low Phosphates | | 10 | | 100 | |
| 6 | Low NaCl | | 11.8 | 114 | | |
| 7 | High NaCl | | | 154 | | |

Appearance (visual inspection) showed clear content with neither color nor visible particle evolution during the stability study for all storage and formulation conditions.

MFI analysis showed low particle levels for all particle sizes and storage conditions. Particle levels increased with the storage temperatures; however, they remained low after 3 months at +40° C. and 6 months at +25° C. No differences between formulation conditions were reported. Results are shown in Table 24 for long-term storage conditions and Table 25 for accelerated and stress storage conditions.

TABLE 24
MFI results in long-term storage conditions

| | | | Time Points | | | | |
|---|---|---|---|---|---|---|---|
| Formulation | Acceptance Criteria | | T0 | T2M | T3M | T6M | T12M |
| Formulation 1 | Particles >2 μm | Report results | 2188 | 4091 | 2548 | 3714 | 1998 |
| | Particles >10 μm | | 28 | 39 | 40 | 39 | 23 |
| | Particles >25 μm | | 2 | 1 | 5 | 2 | 0 |
| Formulation 2 | Particles >2 μm | | 1833 | 1484 | 3042 | 4137 | 1674 |
| | Particles >10 μm | | 26 | 18 | 89 | 31 | 20 |
| | Particles >25 μm | | 4 | 1 | 9 | 4 | 0 |
| Formulation 3 | Particles >2 μm | | 1901 | 1449 | 1207 | 2706 | 1539 |
| | Particles >10 μm | | 31 | 14 | 21 | 22 | 11 |
| | Particles >25 μm | | 3 | 0 | 3 | 0 | 0 |
| Formulation 4 | Particles >2 μm | | 5938 | 6439 | 6487 | 5350 | 5105 |
| | Particles >10 μm | | 38 | 80 | 35 | 55 | 32 |
| | Particles >25 μm | | 4 | 2 | 3 | 7 | 0 |
| Formulation 5 | Particles >2 μm | | 1071 | 2678 | 2977 | 2408 | 3366 |
| | Particles >10 μm | | 22 | 20 | 40 | 13 | 27 |
| | Particles >25 μm | | 3 | 0 | 5 | 4 | 5 |
| Formulation 6 | Particles >2 μm | | 2294 | 2048 | 2417 | 3556 | 1530 |
| | Particles >10 μm | | 20 | 30 | 32 | 82 | 20 |
| | Particles >25 μm | | 2 | 2 | 4 | 2 | 3 |
| Formulation 7 | Particles >2 μm | | 807 | 2146 | 1684 | 4628 | 1346 |
| | Particles >10 μm | | 12 | 38 | 23 | 20 | 7 |
| | Particles >25 μm | | 2 | 3 | 2 | 2 | 0 |

TABLE 25
MFI results in accelerated and stress storage conditions

| | | | +25° C. | | | | | +40° C. | | | |
|---|---|---|---|---|---|---|---|---|---|---|---|
| Formulation | Acceptance Criteria | | T0 | T1M | T2M | T3M | T6M | T0 | T1M | T2M | T3M |
| Formulation 1 | Particles >2 μm | Report results | 2188 | 4703 | 4154 | 10175 | 8453 | 2188 | 4365 | 7758 | 7692 |
| | Particles >10 μm | | 28 | 42 | 39 | 223 | 45 | 28 | 31 | 166 | 92 |
| | Particles >25 μm | | 2 | 3 | 2 | 6 | 2 | 2 | 2 | 2 | 2 |
| Formulation 2 | Particles >2 μm | | 1833 | 6437 | 4267 | 2026 | 2661 | 1833 | 5910 | 2302 | 3376 |
| | Particles >10 μm | | 26 | 76 | 33 | 34 | 26 | 26 | 34 | 27 | 40 |
| | Particles >25 μm | | 4 | 6 | 1 | 7 | 2 | 4 | 2 | 1 | 3 |
| Formulation 3 | Particles >2 μm | | 1901 | 4170 | 2337 | 2256 | 13389 | 1901 | 5191 | 5389 | 4508 |
| | Particles >10 μm | | 31 | 29 | 16 | 27 | 198 | 31 | 77 | 49 | 64 |
| | Particles >25 μm | | 3 | 4 | 0 | 6 | 9 | 3 | 2 | 2 | 6 |
| Formulation 4 | Particles >2 μm | | 5938 | 6070 | 6411 | 8178 | 3153 | 5938 | 7679 | 11206 | 4244 |
| | Particles >10 μm | | 38 | 38 | 157 | 100 | 32 | 38 | 69 | 72 | 44 |
| | Particles >25 μm | | 4 | 4 | 16 | 6 | 1 | 4 | 4 | 2 | 7 |
| Formulation 5 | Particles >2 μm | | 1071 | 4793 | 3301 | 3175 | 3305 | 1071 | 4187 | 3513 | 4086 |
| | Particles >10 μm | | 22 | 33 | 39 | 68 | 27 | 22 | 41 | 26 | 62 |
| | Particles >25 μm | | 3 | 2 | 0 | 15 | 2 | 3 | 6 | 1 | 5 |
| Formulation 6 | Particles >2 μm | | 2294 | 4069 | 2586 | 4005 | 6220 | 2294 | 6066 | 5452 | 9173 |
| | Particles >10 μm | | 20 | 43 | 34 | 66 | 98 | 20 | 73 | 76 | 96 |
| | Particles >25 μm | | 2 | 2 | 2 | 5 | 8 | 2 | 4 | 4 | 3 |
| Formulation 7 | Particles >2 μm | | 807 | 3876 | 2399 | 3940 | 2544 | 807 | 5897 | 3594 | 3434 |
| | Particles >10 μm | | 12 | 22 | 22 | 36 | 33 | 12 | 36 | 38 | 96 |
| | Particles >25 μm | | 2 | 0 | 1 | 2 | 3 | 2 | 7 | 1 | 9 |

DLS analysis showed no differences between formulation conditions whatever the storage condition.

Capillary isoelectric focusing (cIEF) analysis showed a significant increase of acidic species and decrease of basic species in accelerated and stress storage conditions over time. Acidic species in formulation 3 had higher increase than both formulation 1 in accelerated and stress conditions whereas Formulation 2 has shown the lower increase of acidic species. The pH value therefore has an impact on product degradation. However, these observations were not observed in long-term storage condition where no product degradation was observed over 12 months for all formulation conditions. Results are shown in Tables 26 and 27, below.

TABLE 26
cIEF results in long-term storage condition

| | | | Time Points | | | | |
|---|---|---|---|---|---|---|---|
| Formulation | Acceptance Criteria | | T0 | T2M | T3M | T6M | T12M |
| Formulation 1 | Acidic form (%) | ≤50% | 24.7 | 25.8 | 26.3 | 25.1 | 28.5 |
| | Main peak (%) | ≥30% | 61.5 | 61.0 | 59.9 | 61.4 | 59.1 |
| | Basic form (%) | ≤30% | 13.8 | 13.2 | 13.7 | 13.5 | 12.5 |
| Formulation 2 | | | 23.8 | 24.5 | 25.2 | 24.7 | 27.0 |
| | | | 62.3 | 62.0 | 61.3 | 61.6 | 60.2 |
| | | | 13.9 | 13.5 | 13.5 | 13.7 | 12.7 |
| Formulation 3 | | | 25.1 | 26.6 | 26.7 | 26.1 | 30.5 |
| | | | 61.4 | 60.3 | 60.4 | 61.1 | 56.9 |
| | | | 13.6 | 13.1 | 13.0 | 12.8 | 12.6 |
| Formulation 4 | | | 24.4 | 25.6 | 25.6 | 25.2 | 29.0 |
| | | | 62.2 | 61.1 | 60.8 | 61.5 | 58.1 |
| | | | 13.5 | 13.2 | 13.6 | 13.3 | 12.9 |
| Formulation 5 | | | 24.2 | 25.8 | 25.4 | 25.2 | 29.1 |
| | | | 62.0 | 60.6 | 61.2 | 61.7 | 58.0 |
| | | | 13.8 | 13.6 | 13.4 | 13.1 | 12.9 |
| Formulation 6 | | | 24.2 | 26.2 | 26.5 | 25.8 | 29.6 |
| | | | 62.1 | 59.9 | 59.8 | 61.2 | 57.7 |
| | | | 13.7 | 13.8 | 13.7 | 13.0 | 12.7 |
| Formulation 7 | | | 25.1 | 26.0 | 26.4 | 25.8 | 29.0 |
| | | | 61.1 | 60.6 | 60.1 | 60.8 | 58.1 |
| | | | 13.8 | 13.4 | 13.5 | 13.4 | 12.9 |

TABLE 27
cIEF results in accelerated and stress storage conditions

| | | | +25° C. | | | | | +40° C. | | | |
|---|---|---|---|---|---|---|---|---|---|---|---|
| Formulation | Acceptance Criteria | | T0 | T1M | T2M | T3M | T6M | T0 | T1M | T2M | T3M |
| Formulation 1 | Acidic form (%) | Report results | 24.7 | 31.7 | 36.4 | 40.6 | 51.9 | 24.7 | 67.9 | 84.1 | 91.9 |
| | Main peak (%) | | 61.5 | 55.7 | 52.9 | 48.9 | 39.5 | 61.5 | 26.5 | 12.7 | 7.2 |
| | Basic form (%) | | 13.8 | 12.6 | 10.7 | 10.4 | 8.6 | 13.8 | 5.6 | 3.2 | 0.9 |
| Formulation 2 | | | 23.8 | 29.0 | 32.3 | 35.0 | 43.5 | 23.8 | 59.0 | 76.0 | 84.7 |
| | | | 62.3 | 57.9 | 55.7 | 53.2 | 46.5 | 62.3 | 33.9 | 19.6 | 13.1 |
| | | | 13.9 | 13.1 | 12.0 | 11.8 | 9.9 | 13.9 | 7.1 | 4.4 | 2.3 |
| Formulation 3 | | | 25.1 | 34.0 | 41.4 | 46.4 | 60.4 | 25.1 | 76.0 | 90.3 | 94.7 |
| | | | 61.4 | 53.9 | 48.0 | 44.3 | 32.7 | 61.4 | 20.2 | 8.3 | 4.3 |
| | | | 13.6 | 12.1 | 10.6 | 9.3 | 7.0 | 13.6 | 3.9 | 1.5 | 1.0 |
| Formulation 4 | | | 24.4 | 31.0 | 36.2 | 39.8 | 51.3 | 24.4 | 67.2 | 83.9 | 90.4 |
| | | | 62.2 | 57.1 | 52.3 | 49.1 | 40.4 | 62.2 | 27.0 | 12.9 | 8.0 |
| | | | 13.5 | 12.0 | 11.5 | 11.1 | 8.3 | 13.5 | 5.8 | 3.2 | 1.6 |
| Formulation 5 | | | 24.2 | 30.9 | 36.0 | 39.3 | 50.7 | 24.2 | 67.8 | 83.3 | 90.3 |
| | | | 62.0 | 56.8 | 52.6 | 50.0 | 40.7 | 62.0 | 26.8 | 13.8 | 8.1 |
| | | | 13.8 | 12.3 | 11.4 | 10.8 | 8.6 | 13.8 | 5.5 | 2.9 | 1.6 |
| Formulation 6 | | | 24.2 | 31.9 | 37.3 | 41.9 | 52.8 | 24.2 | 70.2 | 85.8 | 91.7 |
| | | | 62.1 | 55.2 | 51.3 | 47.9 | 38.8 | 62.1 | 24.6 | 11.8 | 7.0 |
| | | | 13.7 | 13.0 | 11.4 | 10.2 | 8.5 | 13.7 | 5.2 | 2.4 | 1.3 |
| Formulation 7 | | | 25.1 | 32.0 | 36.1 | 40.3 | 50.3 | 25.1 | 67.0 | 83.5 | 90.1 |
| | | | 61.1 | 55.4 | 52.5 | 48.6 | 40.8 | 61.1 | 27.6 | 13.6 | 8.2 |
| | | | 13.8 | 12.7 | 11.3 | 11.1 | 8.9 | 13.8 | 5.5 | 2.9 | 1.7 |

Purity was assessed by capillary electrophoresis sodium dodecyl sulfate in non-reduced form (CE-SDS NR) and in reduced form (CE-SDS R). Results in non-reduced conditions showed HMW and fragment increases over time in stress storage condition with higher fragment levels for the formulation condition number 3 (pH 7.4) and lower fragment levels for the formulation 2. In the long-term storage condition, HMW and fragment levels remained stable over time without any differences between formulation conditions up to the 12 month time point. CE-SDS R analysis also showed stability in long-term storage conditions, and total antibody for all formulations was around 98% at all time points.

In summary, the study reporting results over 12 months showed that with respect to macroscopic aggregation, neither particles (visible and sub-visible) nor color and clarity content evolutions have been reported. With respect to microscopic aggregation, neither HMWP nor pH value evolutions have been reported over the stability study for all storage conditions. Moreover, no differences between formulations have been reported. Regarding protein concentration and pH, no evolution protein concentration or pH was observed over time for all formulation condition assessed. In accelerated and/or stress storage conditions differences were observed among the different formulations suggesting that higher pH leads to higher degradation of the product.

1. A stable aqueous pharmaceutical formulation comprising:
(i) an anti-KIR3DL2 antibody that binds to a KIR3DL2 receptor;
(ii) one or more buffers;
(iii) a tonicity agent; and
(iv) a non-ionic surfactant;
wherein the ratio of the antibody concentration to non-ionic surfactant is about 20:0.1 to 40:0.4.

2. The stable aqueous pharmaceutical formulation of claim 1, wherein antibody comprises a
heavy chain variable region (VH) and a light chain variable region (VL), and wherein the VH comprises three complementarity determining regions (CDRs) CDR1, CDR2, and CDR3 having the amino acid sequences set forth in SEQ ID NOs: 6, 7 and 8, respectively and wherein the VL comprises three CDRs, CDR1, CDR2, and CDR3 having the amino acid sequences set forth in SEQ ID NOs: 9, 10 and 11, respectively; or
a VH and VL region comprising the amino acid sequence of SEQ ID NOS: 5 and 3 respectively.

3. (canceled)

4. The stable aqueous pharmaceutical formulation of claim 1, wherein the antibody concentration is at least 15 mg/ml.

5. (canceled)

6. The stable aqueous pharmaceutical formulation of claim 1, wherein the buffer is a phosphate buffer selected from the group consisting of sodium dihydrogen phosphate, disodium hydrogen phosphate, sodium phosphate, or any combination thereof; or
wherein the buffer is a dibasic sodium phosphate and monobasic sodium phosphate.

7. (canceled)

8. The stable aqueous pharmaceutical formulation of claim 1, wherein:
the tonicity agent is selected from a salt, a sugar or sugar alcohol, an amino acid, an alditol, polyethyleneglycol, or any combination thereof; or
the tonicity agent comprises sodium chloride.

9. (canceled)

10. The stable aqueous pharmaceutical formulation of claim 1, wherein
the nonionic surfactant is selected from polyoxyethylene sorbitan fatty acid esters, polyoxyethylene alkyl ethers, alkylphenylpolyoxyethylene ethers, polyoxyethylene-polyoxypropylene copolymer or combinations thereof;
the non-ionic surfactant is polysorbate; or
the non-ionic surfactant is polysorbate 80 at a concentration of 0.02-0.2 mg/ml.

11-13. (canceled)

14. The stable aqueous pharmaceutical formulation of claim 1, wherein the formulation has a pH of about 6.9-7.9, optionally 7.2.

15. (canceled)

16. The stable aqueous pharmaceutical formulation of claim 1, wherein the ratio of the antibody concentration to non-ionic surfactant is about 30:0.2.

17. (canceled)

18. The stable aqueous pharmaceutical formulation of claim 1, wherein the formulation is prepared for intravenous administration.

19-26. (canceled)

27. The stable aqueous pharmaceutical formulation of claim 1, wherein the formulation is:
(i) stored for 6 months to 48 months;
(ii) stored at a temperature above 3° C.; or
(iii) both (i) and (ii).

28-43. (canceled)

44. A method of reducing stress-induced particle formation or aggregation during drug pooling comprising:
mixing a non-ionic surfactant with a stored antibody formulation to provide a stabilized antibody formulation comprising a ratio of antibody to non-ionic surfactant of 20:0.1 to 40:0.4;
the antibody formulation having been stored for at least 6 months;
whereby the stabilized antibody formulation comprises a reduced amount of particles or aggregated antibody after pooling than the stored antibody formulation after pooling with no additional non-ionic surfactant.

45. The method of claim 44, wherein the mixing comprises pumping or passing through a syringe used for IV infusion.

46. The method of claim 44, wherein the ratio of the antibody concentration to non-ionic surfactant is about 30:0.2.

47. The method of claim 44, wherein the antibody comprises a heavy chain and light chain comprising the amino acid sequence of SEQ ID NOS: 1 and 2 respectively.

48. The method of claim 44, wherein the antibody concentration comprises 10 mg/ml to 30 mg/ml, 10 mg/ml to 20 mg/ml, or 12 mg/ml to 18 mg/ml.

49. The method of claim 44, wherein
the non-ionic surfactant is a polysorbate 80; or the non-ionic surfactant is a polysorbate 80 comprising about 0.01 mg/ml to about 0.2 mg/ml.

50. (canceled)

51. (canceled)

52. The method of claim 44, wherein the formulation has a pH of about 6.9-7.9, optionally 7.2.

53. (canceled)

54. The method of claim 44, wherein the formulation is:
(i) stored for 6 months to 48 months;
(ii) stored at a temperature above 3° C.; or
(iii) both (i) and (ii).

55-59. (canceled)

60. The method of claim 44, wherein the stabilized antibody formulation comprises 10-fold less particles or aggregated antibody than the stored antibody formulation after storage for at least 6 months followed by being subjected to shear or interfacial stress, optionally wherein the non-stabilized antibody formulation is a formulation having a ratio of the antibody concentration to non-ionic surfactant of less than 20:0.1.

61-70. (canceled)

71. A method of treating comprising:
administering to a subject in need thereof an effective amount of a pharmaceutical formulation comprising:
(i) an anti-KIR3DL2 antibody that binds to a KIR3DL2 receptor at a concentration of at least 15 mg/ml;
(ii) polysorbate 80 at a concentration of 0.02-0.2 mg/ml; wherein the formulation further comprises:
(a) one or more buffers; and
(b) a tonicity agent.

72. The method of treating claim 71, wherein the subject in need thereof comprises:
(i) malignancies involving CD4+ T cells that express KIR3DL2 receptors;
(ii) cutaneous T-cell lymphoma (CTCL) or peripheral T cell lymphoma (PTCL); or
(iii) malignancies such as Mycosis Fungoides and Sezary Syndrome, and KIR3DL2-expressing autoimmune disorders.

73. (canceled)

74. (canceled)
